SEQUENCE LISTING (30 sequences)

SEQ ID NO: 1 - DNA, 16 bp, Artificial Sequence
  Pst I SSAP adapter primer
gactgcgtacatgcag

SEQ ID NO: 2 - DNA, 313 bp, Zea mays
  Heartbreaker (Hbr7) Miniature Inverted Repeat Transposable Element (MITE) (AF 203730)
gggtctgtttggttcagcttttttctgaccagcttttctgaaaatctggctgtagggagatctggccgtgggaagaatctgagtatcattacgattacgtgtggaggaagataaagttgttcatagggctcatgatctagaaagtgacggattcctactattacaacgactcaaccgattatatgtttatgttaattttggatggtttttgccccaacgaattttatagaagctggctgaaaagctgagtgtttggcagtccgcagcagcttttggtggccagaagctgtcagaagccgaaacaaacagggcc

SEQ ID NO: 3 - DNA, 16 bp, Zea mays
  left flanking (FL) sequence of Hbr7
cggacgcgccagccat

SEQ ID NO: 4 - DNA, 16 bp, Zea mays
  right flanking (FR) sequence of Hbr7
catcctttgctttggt

SEQ ID NO: 5 - DNA, 20 bp, Artificial Sequence
  FL/ME oligonucleotide
cgccagccatgggtctgttt

SEQ ID NO: 6 - DNA, 20 bp, Artificial Sequence
  ME/FR oligonucleotide
aaacagggcccatcctttgc

SEQ ID NO: 7 - DNA, 18 bp, Artificial Sequence
  FL/FR oligonucleotide
gcgccagccatcctttgc

SEQ ID NO: 8 - DNA, 21 bp, Artificial Sequence
  a second form of MITE locus
gcgccagccatcatcctttgc

SEQ ID NO: 9 - DNA, 100 bp, Zea mays
  FL region of Zeon-1 LTR retrotransposon
tgcctatatttgtactatcgatcatattaataatagtacgagatagaatgataacaatacacatgactagaatatgttattttttctaacctgaaaggta

SEQ ID NO: 10 - DNA, 100 bp, Zea mays
  FR region of Zeon-1 LTR retrotransposon
aggtacccactggtagccctaataataattctagtcggtgtagggacaagttgtgctacggtcaagagaggggaagcaaaatggccttttatcctgatga

SEQ ID NO: 11 - DNA, 49 bp, Zea mays
  right end of the Zeon-1 LTR
tgttgggggccttcggcttccgaaggtcctcaaaaacaagatttaactg

SEQ ID NO: 12 - DNA, 49 bp, Zea mays
  right end of the Zeon-1 LTR
tgtgttgccttgttcttaattcatagcatttgagaacaagtccccaaca

SEQ ID NO: 13 - DNA, 26 bp, Zea mays
  FL/ME joint
ctaacctgaaaggtactgttgggggc

SEQ ID NO: 14 - DNA, 31 bp, Zea mays
  ME/FR joint
aagtccccaacaggtacccactggtagccct

SEQ ID NO: 15 - DNA, 20 bp, Artificial Sequence
  ME/FR oligonucleotide
tgaaaggtactgttgggggc

SEQ ID NO: 16 - DNA, 21 bp, Artificial Sequence
  ME/FR oligonucleotide
gtccccaacaggtacccactg

SEQ ID NO: 17 - DNA, 22 bp, Artificial Sequence
  FL/FR oligonucleotide
ctgaaaggtacccactggtagc

SEQ ID NO: 18 - DNA, 32 bp, Artificial Sequence
  primer 7286
ggaattcatagcatggataataaacgattatc

SEQ ID NO: 19 - DNA, 28 bp, Artificial Sequence
  (CTC)9C
ctcctcctcctcctcctcctcctcctcc

SEQ ID NO: 20 - DNA, 1767 bp, Hordeum vulgare
  sb17, polymorphic fragment present in spring barley accession
ggaattcatagcatggataataaacgattatcatgatctaagaaatataataataactaatttattattgcctctagggcatatttccaacaccacttcctcgtgctcctcctcaacttcgaggagggaggaagccgccctcccgccgtcagtgcacttcctcgtgctcctcctcaaaatcctgtgaggctttgcttctccccttcccctctgttccacaatgttttttgtaatttttgcccatgatgttgcttgcacggatcaaaaaaatcatatcatctgttggtactttgtccgttgtgtgttttgattttgtgattttcagtgcattgtttcctgattaacatgaatttagttttatacatcaccttaattttgattaattactgaccatggtgagcaagatctaaacaacaagaaatgcacttattaacttgacattgttaattaaaaaaatttgatgaagcacagacttatttcagcaacagtctctgcctttgcatgtcagttaatggatctggcacctttttgtacaaatcaatggatatgacacttagtgttatggattttatgaacaactcacaaattaacgtcattgatgtgtatgatatgtatgcatacttgaacattatgatatgcgtgtatactagcatggtagtaacttgaatgcatcagtgttcgtgccatagagttgttttccgcatccttcctacgcgcgaccaaaaaatcaacccgctcgagaaaacagtccactaaaataaaaaatagacccatgacccacgaacacatgccccttccttatcaaaggacaacctcgttcctcaaaattttccaaccgaacccaccttccttttccgcatgtgccacccacatcgtagccctctctcgcacgcatgtgccgctcgtcctagacggttgctaccgcctctatcggttctcgacctcccgaaggacgcatccatcacgcgcggtcaaggccctgccataggccatggttagtgctgccatccactcatctcctctatacaaccccttctctctcggatccaggctggccaacggtgtcgacccccaattgaagtggccccaccttcctttcctcgtgcgcctcagcgccatttccaggtacgtatcatagcgcaagcgcatgggctgacagcgtccgtcgccgagccttctccaaggatggcagtccaccacaccgtgtgccactgcatcgagttggtagccaccatcgccgcccagtccatctactggtgagacgcgagatctatccacccagttctgcatccatcaggcacccaaccaccaggtatgggccactgctattattatatttctcttctgatttagtcggagatgttgctgttgtgtttgcagtgtgagagagcagaaaggactgtgagagtgcaggggtggtatgatcacgacccaatgtcatggcagtagaggaggcaacagatgtcgaggaggaggaaaggcacatgagctgcggcaggggaggtcgaggaggaggaggaggagaggtatgtgctcggcggcaaccgggtcactgtggtaccaagcacaacctgatccagaacagtctcgcgctctttctgacatgatagacataacctgcacataggttatatatttttctaaagattaattttttttccgacactaattagaattagccaaaatagcgatcatgtcttattagtctcaatattgaattttgcatttgtttcaatattacacaattcacttttggtaaatgcatgc

SEQ ID NO: 21 - DNA, 3186 bp, Hordeum vulgare
  wb17, sequence of winter barley lacking BARE-1 insertion
gcatgcagaaaaaaaaaacaaatctggagaaaacgttcagaatgcgacacgatgcggcggctgaaaacgtgtcaagtgactacacgtgatagtgatcattgagaaacttccaaaagagtgattgctaactagttgttctcataagtcacttataatgaaatggtaactcccctggccggcaatgtcctcccatagccggcccattagcctgtttccaatagcttgctgtccctcgtgcatttcaataattgtgtttggacgctgtaggtccggttttttcttatgttgttcaatttttttgtcattttcgtttttcttttctgtgtttttgtttattggtttttaccggttatttagtgtcactttaatttcataatttcaccatcatatttttatttatttttgtcgtttttatgttcttttttaattgggtgtcctttcatattttattatttttatcattcttatttttctttactatttcactgctttccatatgtttctttggtctttgggtttcttcattttgttttctcttttcgttttctcttttcctacacatgtgtacatgctaggaccagtttttatgcatgttttactttgcctaaatacaagacaaatatttccctagaatatttgttattgtacctattttatatatattttttgttttctgtatgcaatataacatctctactattaaagaggggtctgtcgtcgtcgtgatggttcgacttcgttcgattccctcctagctcctcccttccacgttctcccaccaattttttttcaatcattcgatcccttcgaaaaccgctctctcccattctctttctccaccgcttcgctcaccttcaacccactccccttcctgctcctccccgccagatgcaccccctcctccctgccggatgcaccccctcctccccgccagatgcacccctcctctcctctcctctgccgcccacccagaggacaaccaccaattccttccttcacctccccttctcgtgccccatccaccaccggatccgatcattgcagcaggtggcccgacgcccgtgactgcaccgtccatctcatctcgcctgtgcaggtacttcccttatttcccctccatgccatctctcaccaccaatttccctcacctctcttaccctatttccagatctggaccgtcaaccaccttctcccggaaccaccgtgtcctcggaacgagcaggacaagaggagaggaggcaggagtgcgacgtccgccggcgacctggccatcctcctggatctcaccaggggaggatggagcgagcatggcaatagtaggagaggtgggaacagggcgacgtctgacggcgacctgatcatcctcctggatctcgctggcaacctcctggaggccgcccgcccaccgtcagtgtggggcccacgacaccacttcctcgtgctcctcctcaacacggaggagggagaagggaggaggccgcccgcccgccgtaagtgcggggcccacggcaccacttcctcgtgctcctcctcaacttcgaggaaggaggaagccacccgcccgccgtcagtgcacttcctcgtgctcctcctcaaaatcctgtgaggctttgcttctccccttcccctctgttccacaatgttttttgtaatttttgcccatgatgttgcttgcacggatcaaaaaaatcatatcatctgttggtactttgtccgttgtgtgttttgattttgtgattttcagtgcattgtttcctgattaacatgaatttagttttatacatcaccttaattttgattaattactgaccatggtgagcaagatctaaacaacaagaaatgcacttattaacttgacattgttaattaaaaaaatttgatgaagcacagacttatttcagcaacagtctctgcctttgcatgtcagttaatggatctggcacctttttgtacaaatcaatggatatgacacttagtgttatggattttatgaacaactcacaaattaacgtcattgatgtgtatgatatgtatgcatacttgaacattatgatatgcgtgtatactagcatggtagtaacttgaatgcatcagtgttcgtgccatagagttgttttccgcatccttcctacgcgcgaccaaaaaatcaacccgctcgagaaaacagtccactaaaataaaaaatagacccatgacccacgaacacatgccccttccttatcaaaggacaacctcgttcctcaaaattttccaaccgaacccaccttccttttccgcatgtgccacccacatcgtagccctctctcgcacgcatgtgccgctcgtcctagacggttgctaccgcctctatcggttctcgacctcccgaaggacgcatccatcacgcgcggtcaaggccctgccataggccatggttagtgctgccatccactcatctcctctatacaaccccttctctctcggatccaggctggccaacggtgtcgacccccaattgaagtggccccaccttcctttcctcgtgcgcctcagcgccatttccaggtacgtatcatagcgcaagcgcatgggctgacagcgtccgtcgccgagccttctccaaggatggcagtccaccacaccgtgtgccactgcatcgagttggtagccaccatcgccgcccagtccatctactggtgagacgcgagatctatccacccagttctgcatccatcaggcacccaaccaccaggtatgggccactgctattattatatttctcttctgatttagtcggagatgttgctgttgtgtttgcagtgtgagagagcagaaaggactgtgagagtgcaggggtggtatgatcacgacccaatgtcatggcagtagaggaggcaacagatgtcgaggaggaggaaaggcacatgagctgcggcaggggaggtcgaggaggaggaggaggagaggtatgtgctcggcggcaaccgggtcactgtggtaccaagcacaacctgatccagaacagtctcgcgctctttctgacatgatagacataacctgcacataggttatatatttttctaaagattaattttttttccgacactaattagaattagccaaaatagcgatcatgtcttattagtctcaatattgaattttgcatttgtttcaatattacacaattcacttttggtaaatgcatgc

SEQ ID NO: 22 - DNA, 23 bp, Artificial Sequence
  FR/ME oligonucleotide
tatttccaacacccacttcctcg

SEQ ID NO: 23 - DNA, 266 bp, Artificial Sequence
  ME/FL flanking region
gcccaccgtcagtgtggggcccacgacaccacttcctcgtgctcctcctcaacacggaggagggagaagggaggaggccgcccgcccgccgtaagtgcggggcccacggcaccacttgttggggaacgtcgcatgggaaacaaaaaaattcctacgcgcacgaagacctgtcatggtgatgtccatctatgagggggatttcaaatctacgtacccttgtagatcgcataacagaaatgttaataaacgcggttgatgtagtggaa

SEQ ID NO: 24 - DNA, 46 bp, Artificial Sequence
  FL/ME
gtaagtgcggggcccacggcaccacttgttggggaacgtcgcatgg

SEQ ID NO: 25 - DNA, 50 bp, Artificial Sequence
  ME/FR
cctctagggcatatttccaacaccacttcctcgtgctcctcctcaacttc

SEQ ID NO: 26 - DNA, 21 bp, Artificial Sequence
  FL/ME oligonucleotide
cacggcaccacttgttgggga

SEQ ID NO: 27 - DNA, 22 bp, Artificial Sequence
  FR/FL oligonucleotide
agcacgaggaagtggtgccgtg

SEQ ID NO: 28 - DNA, 21 bp, Artificial Sequence
  Description of Artificial Sequence -
cacggcaccacttcctcgtgc

SEQ ID NO: 29 - DNA, 22 bp, Artificial Sequence
  Description of Artificial Sequence -
gaggaagtgggtgttggaaata

SEQ ID NO: 30 - DNA, 22 bp, Artificial Sequence
  Description of Artificial Sequence -
ctccttcaccctgttggaaata

(19) United States
(12) Patent Application Publication
(10) Pub. No.: US 2003/0170705 A1
(43) Pub. Date: Sep. 11, 2003

(54) Method and test kit for demonstrating genetic identity

(76) Inventors: Alan Howard Schulman, Helsinki (FI); Lars Goran Paulin, Helsinki (FI)

Correspondence Address: John Dodds, 1707 N St. NW, Washington, DC, 20036, US

(21) Appl. No.: 10/351,934

(22) Filed: Jan. 27, 2003

(30) Foreign Application Priority Data
Jan. 30, 2002 (FI) FI 20020176

(51) Int. Cl. C12Q 1/68

(52) U.S. Cl. 435/6

(57) ABSTRACT

The present invention is related to a method and a test kit for demonstrating genetic identity, genetic diversity, genomic variations or polymorphisms, especially allelic variations, and also biodiversity within a defined population pool, with co-dominant scoring. The method and the test kit apply mobile elements (MEs) and are based on the use of one or more sets of optionally paired or parallel oligonucleotides, which are attached to a solid support. Each oligonucleotide sequence represents an insertion site junction of a mobile element (ME). The invention is also related to the use of the method and the test kit for phylogenetic studies, parenthood determinations, genotyping, haplotyping, pedigree analysis, forensic science, human medical diagnostics and in plant and animal breeding by demonstrating genetic identity, genetic diversity, genomic variation or polymorphism and particularly co-dominant scoring.

This is a non-provisional patent application claiming the benefit of an earlier filing date of a patent application FI120020176 filed on Jan. 30^{th}, 2002.

A Sequence Listing according to 37 C.F.R. section 1.821 (c) is attached. Attached hereto is a 3 ½″ disk containing the Sequence Listing in computer readable form in accordance with 37 C.F.R. section 1.821 (e). The content of the sequence listing information recorded in computer readable form is identical to the written sequence listing and includes no new matter.

### TECHNICAL FIELD OF THE INVENTION

The present invention is related to a method and a test kit for demonstrating genetic identity, genetic diversity, genomic variations or polymorphisms, especially allelic variations, and also biodiversity within a defined population pool, with co-dominant scoring. The method and the test kit apply mobile elements (MEs) and are based on the use of one or more sets of optionally paired or parallel oligonucleotides, which are attached to a solid support. Each oligonucleotide sequence represents an insertion site junction of a mobile element (ME). The method and the test kit are useful for genetic identity determination, phylogenetic studies, parenthood determinations, genotyping, haplotyping, pedigree analysis, forensic science, human medical diagnostics, and in plant and animal breeding.

### BACKGROUND OF THE INVENTION

The genome of a given individual (e.g. human, animal, bacterial, plant etc.) within a given population is for the main part unique, unless highly inbred or clonally or asexually propagated. The uniqueness of a given genome is determined largely by the sequence of DNA contained, therein. Given that differences in genome uniqueness between individuals reflect differences in DNA sequence, then DNA sequence variation can be used to discriminate individuals from each other i.e. genotyping distinguishes phenotypes. Detecting DNA sequence variation can be achieved using a variety of laboratory-based procedures each with their own inherent limitations and advantages; it is a balance between these two extremes that determines the usefulness of the method chosen. Whatever the approach used the objective remains the same: to detect DNA sequence variation and to use that information to discriminate individuals from each other. The profile of DNA sequence variation that discriminates one individual from another is termed a “DNA fingerprint”. As a technique, DNA fingerprinting has an immense range of applications including, but not restricted to, forensic identification, phylogenetic studies, parenthood determination, forensic science, human medical diagnostics, pedigree analysis and animal and plant breeding.

By way of example, traditional plant breeding relies on the expertise of the “breeders eye” to identify and to follow the inheritance of given traits, which are introduced from a donor plant into a recipient variety, by crossing and back-crossing until the unwanted genetic background from the donor has been eliminated. The number of backcrossing steps required to achieve this goal of the breeding program typically requires several years' effort. To accelerate the breeding program, highly selective marker assisted selection (MAS) and DNA fingerprint profiling processes can be applied; these processes are carried out in the laboratory using molecular biological techniques. There are numerous DNA markers that can be used for DNA fingerprinting. Each marker has its own inherent advantages and disadvantages.

Restriction Fragment Length Polymorphism (RFLP) (Botstein, et al., Am. J. Hum. Genet. 32: 314-331, 1980; WO 90/13668) is one of the pioneering marker systems. The resolving power of RFLPs allows identification of heterozygous and homozygous states.

In other words, RFLPs are co-dominant markers. There are, however, several distinct disadvantages associated with the use of RFLPs for routine marker assisted selection (MAS) and DNA fingerprinting. RFLP analysis is extremely labour intensive involving lengthy protocols and the use of high-energy radioactive isotopes, and the development costs are high. Furthermore, the number of markers that can be analysed per assay is typically only one or two.

Since the introduction of RFLPs many alternative markers have been developed including Single Nucleotide Polymorphism (SNPs; Kwok, et al., Genomics 31: 123-126, 1996), Randomly Amplified Polymorphic DNA (RAPD; Williams, et al., Nucl. Acids Res. 18: 6531-6535, 1990), Simple Sequence Repeats (SSRs; Zhao & Kochert, Plant Mol. Biol. 21: 607-614, 1993; Zietkiewicz, et al. Genomics 20: 176-183, 1989), Amplified Fragment Length Polymorphism (AFLP; Vos, et al., Nucl. Acids Res. 21: 4407-4414, 1995), Short Tandem Repeats (STRs) or Variable Number of Tandem Repeats (VNTR), and microsatellites (Tautz, Nucl. Acids. Res. 17: 6463-6471, 1989; Weber and May, Am. J. Hum. Genet. 44: 388-396, 1989).

Among the systems applying markers the Sequence-Specific Amplified Polymorphism method (SSAP; Waugh, et al., Mol. Gen. Genet. 253: 687-694, 1997), the Retrotransposon Microsatellite Amplified Polymorphism (REMAP) system and Inter-Retrotransposon Amplified Polymorphism (IRAP) system can be mentioned. The REMAP and IRAP (Kalendar, et al., Theor. Appl. Genet. 98: 704-711, 1999) systems are considerably less time consuming, universally applicable and more informative than for example the conventional RFLP system, but it is to be noted that REMAP and IRAP are not co-dominant markers and generally can not therefore be used to distinguish between heterozygous and homozygous genotypes.

Retrotransposon-based insertion polymorphism (RBIP) (Flavell et al., Plant J 16:643-650, 1998) is a retrotransposon-based marker system. It is most analogous to microsatellite marker systems in that a single site is analysed per primer pair and that the primers correspond to sequences flanking a variable region, which generates the allelic variability. Accordingly RBIP differs from SSAP, IRAP, and REMAP which reveal multiple but anonymous sites with each PCR amplification reaction, and is unlike microsatellite systems, which detect not only allelic variation in a set of simple sequence repeats (SSRs) between the primers, but the presence or absence of a retrotransposon at that position. Marker molecules and systems discussed above, are disclosed for instance in WO 93/06239, WO 00/35418, EP 967291, WO 01/27321, U.S. Pat. No. 6,114,116, WO 95/11995 and WO 99/67421.

The above list of markers, marker systems as well as patents or patent applications is non-exhaustive. Despite the multitude of available or suggested systems, it is also evident that each system has its own inherent advantages and disadvantages, no system being ideal for all purposes. One of the problems with a marker system applying PCR and genomic elements is the fact that the capacity to amplify the whole genomic element sometimes fails and minor errors are duplicated, which reduces resolution. Therefore, a need to provide alternative systems, which are sufficiently effective for the demands of, for example, modern breeding techniques, still exists.

A clear need exists for an alternative marker system including a method and a test kit, which is universal in its application, provides robust, reproducible generation of marker pattern with an inexpensive and technically straightforward detection system.

### BRIEF SUMMARY OF THE INVENTION

The present invention is related to a method and a test kit for demonstrating genetic identity, genetic diversity, genomic variations or polymorphisms especially allelic variations, and also biodiversity within a defined population pool based on detection of the presence or absence of mobile elements (MEs) and their respective insertion site junctions across the whole range of genotypes in a population pool. The method, which applies a solid support with attached oligonucleotide sequences is useful for genetic identity determination, phylogenetic studies, parenthood determinations, genotyping, haplotyping, pedigree analysis, forensic science, human medical diagnostics and in plant and animal breeding. The method allows detection of changes in certain genomic positions by recording the presence or absence of mobile elements (MEs). A result with a desired level of resolution within a population pool/in a pool of genotypes is achieved with the method and test kit of the present disclosure, which enable the use of sheared unlabeled sample DNA for the hybridization. This means that special precautions with preservation of the DNA specimen are unnecessary.

The fact that unlabeled specimen DNA is used for the hybridization means that DNA purity is not as important as it is when the specimen DNA itself is labelled. Furthermore, reference specimens can be easily maintained and used without the special precautions that are needed for labelled DNA. Large numbers of sample DNAs can be processed more cheaply because only shearing is required (after DNA extraction).

The method and the test kit for detection of hybridized oligonucleotides in the detection step are not specific to the sample DNA itself, but are based on a general method relying on one or more means for distinguishing the hybridized forms from the unhybridized oligonucleotide forms. As such it is given to standardization and automation independent of the particular sample investigated or its quality.

Due to the relative simplicity of use, the method and the test kit make the invention applicable for in-house use by, for example, breeders. The method and the test kit of the present invention provide a straightforward and practical approach for the breeder, who prefers to monitor and take responsibility for their own in-house quality control.

A specific advantage of the method and the test kit of the present invention is that they can be used for reliable discrimination between the heterozygous and homozygous state in back-crossed progeny for a given gene of interest without having to determine the zygosity state by retrospective conventional screening of corresponding (self-fertilized) generations after back-crossing. This has the major cost benefit that the breeding program can be considerably shortened.

Accordingly, the present invention is related to a method and a test kit, which enable the determination of genetic identity, genetic diversity and genetic variation such as genomic variations or polymorphism, especially allelic variations, and also biodiversity within a defined population pool, with co-dominant scoring. The present invention applies mobile elements (MEs) and is based on the use of sets of optionally paired or parallel oligonucleotides, which are attached to a solid support. Each oligonucleotide sequence represents either a full or an empty integration site of a mobile element (ME) and is composed of two parts, which represent either a terminal end of a mobile element (ME) or a flanking region or flanking regions of said mobile element (ME). The oligonucleotide sequence which detects a full integration site comprises partly a flanking region of a defined mobile element (ME) and partly the terminus of said mobile element (ME) and the oligonucleotide sequence which detects an empty integration site comprises both left and right flanking regions on each side of the integration site. The method and the test kit are useful for genetic identity determination, phylogenetic studies, parenthood determinations, genotyping, haplotyping, pedigree analysis, in forensic science, for human medical diagnostics and to provide assured and accelerated breeding, especially providing co-dominant scoring.

In the method, unlabeled, optionally fragmented single stranded oligonucleotide sequences representing the total DNA of a sample are allowed to hybridize with more than one set of optionally paired or parallel oligonucleotide sequences which as described above are composed of two elements or parts, which are of varying length. In other words, each set of oligonucleotides represents a defined genomic position or integration site.

The different steps of the method including hybridization, post-hybridization treatment, recording of hybridization and scoring are automated in the preferred embodiment of the present invention.

The present invention is also related to a test kit for demonstrating particularly with co-dominant scoring genetic diversity, genetic identity, genomic variations or polymorphisms, especially allelic variations, and biodiversity within a defined population pool. More specifically, the test kit comprises a solid support, which may be a membrane, filter, slide, plate, chip, dish, etc. Even microwells on a microtiter plate are suitable as solid supports. The solid support can be composed of a material such as glass, plastics, nitrocellulose, nylon, polyacrylic acids, silicons, etc. The test kit may contain optional reagents including labels, washing buffers, end protection reagents and/or instruction for use.

The test kit is characterized by comprising more than one set of optionally paired or parallel oligonucleotide sequences. In its simplest form the test kit therefore comprises two different single oligonucleotides, one for an empty integration site and one for a full integration site, wherein each oligonucleotide is capable of recognizing a specific, defined insertion site junction of a mobile element (ME) as well as the presence or absence of the mobile element (ME) in said insertion site junction. However, one skilled in the art would realize that in order to obtain sufficiently informative information of the genetic diversity in a population pool more complex systems must be provided.

Therefore, in preferred embodiments of the invention more sets of oligonucleotides are required. It can be calculated that in order to obtain optimal fingerprinting or mapping results in a diploid organism with seven chromosome pairs, the minimum of oligonucleotide sets should be about 70-80. For organisms having more chromosomes, more sets of oligonucleotides are desirable. However, there are no upper limits for the number of oligonucleotide sets.

One pair is sufficient and the upper limit is provided by the presence of available, characterized DNA sequences especially mobile elements (MEs) for the subject to be identified. In other words, the number of oligonucleotide sets depends on the availability of informative flanking sequence DNA pairs and, in respect of marker assisted selection (MAS), the location of the sequence pairs in relation to known genes of interest.

The information obtainable by the present invention can be further improved by using not only several sets of oligonucleotides, but by providing two or more optionally parallel or paired oligonucleotide sets for each mobile element (ME) to be determined. Said optionally paired or parallel oligonucleotide combinations may for example be designed as follows:

left flanking region (FL)+terminal end of mobile element (ME) combined with left flanking region (FL)+right flanking region (FR);

right flanking region (FR)+terminal end of mobile element (ME) combined with left flanking region (FL)+right flanking region (FR);

left flanking region (FL)+terminal end of mobile element (ME), right flanking region (FR)+the other terminal end of the mobile element (ME) combined with left flanking region (FL)+right flanking region (FR).

The oligonucleotides may be prepared from any of the complementary strands as both strands of the DNA sample will be present in single stranded form before the hybridization reaction takes place.

The oligonucleotide sequences of the test kit can optionally be end-protected and the test kit, with the oligonucleotides attached to the solid support, is reusable, when reversible development and hybridization recording treatments are used.

The present invention also allows the use of the method and the kit for distinguishing any organism differing by at least one mobile element (ME) in any given genomic position or at least one flanking region in any given genomic position. Also included is the use of the method and the test kit for genetic identity determination, phylogenetic studies, parenthood determinations, genotyping, haplotyping, pedigree analysis, forensic science, human medical diagnostics and in plant and animal breeding.

### BRIEF DESCRIPTION OF THE DRAWINGS

FIG. 1 shows different types of mobile elements (MEs).

FIG. 1A depicts DNA-mediated transposons, which constitute the so-called Class II elements and move by cutting and pasting of a chromosomal segment to a new location. Class II elements include both autonomous (self-mobilizing) and non-autonomous elements; non-autonomous elements include Miniature Inverted Repeat Transposable Elements (MITEs), which are highly-deleted versions of mobile elements (MEs). Abbreviation: ds, double stranded.

FIG. 1B depicts RNA-mediated transposable elements, retrotransposons, or Class I elements, which do not excise as do Class II elements but instead make daughter copies through the process of reverse transcription and which are then inserted into a new genomic position in the genome. Abbreviations: ds, double stranded; rev., reverse; AAAA, poly(A) tail.

FIG. 1C depicts Long Terminal Repeat (LTR) retrotransposons. The LTR retrotransposons represent one of the two major groups of Class I transposable elements. The group includes both gypsy-like (a) and copia-like (b) elements, the former being more retroviral like in structure and sequence. The domains of the LTR, U3, R and U5 are shown. Abbreviations: PBS, primer binding site; GAG, capsid protein; AP, aspartic proteinase; IN, integrase; LTR, long terminal repeat; RT, reverse transcriptase; RH, ribonuclease H; PPT, polypurine tract.

FIG. 1D depicts non-Long Terminal Repeat (non-LTR) retrotransposons. The non-LTR retrotransposons include Long Interspersed Elements (LINEs) (a) and Short Interspersed Elements (SINEs) (b). For details of the classes of retrotransposons and the products they encode see Kumar & Bennetzen, Annu Rev. Genet. 33: 479-532, 1999. Abbreviations: GAG, capsid protein; RT, reverse transcriptase; RH, ribonuclease H; UTR, untranslated region; EN, endonuclease, (A)n, 3′ polyadenylation sequence.

FIG. 2 schematically illustrates alternative arrangements of an oligonucleotide(s) corresponding to the left flank (FL) and/or right flank (FR) and/or the corresponding end of a mobile element (ME) attached by a linker(s) to a solid support. Abbreviations: ME, mobile element; FL, left flanking region; FR, right flanking region. It is to be noted that each oligonucleotide shown in the Figures represent a multitude of identical oligonucleotides.

FIG. 2A schematically illustrates one alternative arrangement of a single oligonucleotide attached by a linker to a solid support representing a single mobile element (ME) insertion site in genomic DNA (a). Different types of oligonucleotides can be used and are herein proposed, two oligonucleotides corresponding to the mobile element (ME) insertion site junction, wherein the left or right flank and the corresponding end of the mobile element (ME) are shown (b), and an insertion junction with both the left and right flanks but with the site for the mobile element (ME) unoccupied (c).

FIG. 2B schematically illustrates the arrangement of separate oligonucleotides representing the left flank (FL) and/or right flank (FR) and/or the corresponding end of a mobile element (ME) attached by separate linkers to a solid support. Three arrangements are proposed, two corresponding to the mobile element (ME) insertion site junction (a) and (b), and one representing the unoccupied site of a mobile element (ME) insertion site event (c). Even if there seems to be a gap between the separate oligonucleotides, it is essential that the oligonucleotides are situated closely enough so that the genomic sample DNA can hybridize with both oligonucleotides in the case of a full or empty insertion site.

FIG. 2C schematically illustrates the arrangement of separate oligonucleotides representing the left flank (FL) and/or right flank (FR) and/or the corresponding end of a mobile element (ME) attached by complementary oligonucleotide (complementary base pairing) extensions attached to separate linkers attached to a solid support. Three arrangements are proposed, two corresponding to the mobile element (ME) insertion site junction (a) and (b), and one representing the unoccupied site of a mobile element (ME) insertion site event (c).

FIG. 3 schematically illustrates the concept of the present invention including a solid support. Abbreviations: ME, mobile element; FL, left flanking region; FR, right flanking region.

FIG. 3A schematically shows the solid support (grey bar) with three oligonucleotides immobilized on it. The linkers are shown as black ovals. Three kinds of oligonucleotides are shown as examples: left flank/right flank (FL/FR) (a), with the left flank (FL) and right flank (FR) segments shaded, respectively with differing stripe patterns; left flank/mobile element (FL/ME) (b), the mobile element (ME) shown as a hatched box; and mobile element/right flank (ME/FR) (c). The small circles at the ends of the oligonucleotides are extensions of one or more bases added to the oligonucleotide and not matching the flanking sequences. The solid support can be any solid support, including beads, and the three oligonucleotides do not need necessarily to be immobilized to the same support. The three oligonucleotides shown represent the three oligonucleotides for one given genomic position.

FIG. 3B schematically shows total DNA (a; squiggly line); b, c, d and e represent different DNA fragments sheared from total DNA and representing the genomic equivalents of the oligonucleotides shown as in FIG. 3A, together with the flanking sequence (squiggles). The flanking sequence includes an internal mobile element (ME) sequence shown as a hatched box.

FIG. 3C schematically shows oligonucleotides on a solid support, as in FIG. 3A, hybridized to fragments of genomic DNA. In this particular example, only the empty site [left flank/right flank (FL/FR)] oligonucleotide matches the genomic DNA completely (a). For an oligonucleotide comprising left flank/mobile element (FL/ME), only the mobile element (ME) matches for one particular fragment of sheared genomic DNA (b); for mobile element/right flank (ME/FR), only the right flank (FR) segment matches in another case (c). In other cases different patterns would be detected.

FIG. 3D schematically shows the washing step carried out, removing only the partially to the solid support attached oligonucleotides hybridized genomic DNAs.

FIG. 3E schematically shows the detection step carried out. The detectable label, incorporated by extension of the hybridized DNA, is shown as black circles.

FIG. 3F schematically shows the scoring of the detection reaction. Oligonucleotide left flank/right flank (FL/FR) (a) represents the empty site, and gives a positive signal. Oligonucleotides left flank/right flank (FL/ME) (b) and mobile element/right flank (ME/FR) (c) represent the full site, and both give no signal. Hence, the site is confirmed as empty.

FIG. 4 shows for comparative reasons only the prior art Retrotransposon-based Insertion Polymorphism (RBIP) method. The method relies on detection of the presence or absence of an insertion of a mobile element (ME) at a particular genomic position (Flavell, et al., Plant J. 16, 643-650, 1998). Abbreviations: ME, mobile element; FL, left flanking region; FR, right flanking region.

FIG. 4A demonstrates PCR at the empty site using primers from the left flank (FL) and right flank (FR) of a mobile element (ME) insertion, generating a product (below).

FIG. 4B demonstrates PCR reactions from the genomic position following a mobile element (ME) insertion. The left flank (FL) and right flank (FR) primers are combined with primers pointing to the left (L) and right (R), with respect to the sense direction of the mobile element (ME). PCR amplification with the combination FL+FR generally fails to yield a product because, in this example, the distance between the PCR primers, determined by the size of the inserting mobile element (ME), is great (N.B. the absence of a corresponding PCR product is shown as a dotted line below). The combinations FL+L and FR+R will yield products for this full genomic position, whereas they will not for the empty genomic position in (FIG. 4A). As described in the literature (Flavell et al., Plant J 16:643-650, 1998), RBIP is scored by separating the PCR products on an agarose gel. Alternatively, the PCR reaction products can be placed onto an appropriate filter and then hybridized in separate reactions using oligonucleotides from the amplified part of the mobile element (ME) or flanking sequence, as appropriate.

FIG. 5 depicts how mobile element (ME) insertion polymorphism can discriminate between heterozygous and homozygous states.

FIG. 5A shows homologous chromosomes bearing one (heterozygous state) or two (homozygous state) mobile elements (MEs) at the same genomic position.

FIG. 5B shows inverse PCR using primers designed to the mobile element (ME) identifying plant genomic DNA sequences (dotted lines) that immediately flank the mobile element (ME). Note that in the heterozygous state the mobile element (ME) is absent on one of the homologous chromosomes.

FIG. 5C shows long range PCR using inward facing primers designed to the left and right mobile element (ME) flanks amplifying either one or two PCR products (a or b) depending on whether or not the mobile element (ME) is present on one or both homologous chromosomes.

FIG. 5D shows gel electrophoresis separating the PCR amplified product(s) according to size (in this example, ‘a’ alone or ‘a’ and ‘b’) thereby resolving the heterozygous state from the homozygous state.

FIG. 6 schematically shows a modification of the present invention including a solid support. The detection method is different as compared with the schematic illustration in FIG. 3. Abbreviations: ME, mobile element; FL, left flanking region; FR, right flanking region.

FIG. 6A schematically depicts the solid support (grey bar) with three oligonucleotides immobilized on it. The linkers are shown as black ovals. Three kinds of oligonucleotides are shown as examples: left flank/right flank (FL/FR) (a), with the left flank (FL) and right flank (FR) segments shaded, respectively with differing stripe patterns; left flank/mobile element (FL/ME) (b), the mobile element (ME) shown as hatched box; and mobile element/right flank (ME/FR) (c). The solid support can be any solid support, including beads, and the three oligonucleotides do not need necessarily to be immobilized on the same support. The three oligonucleotides shown represent the three oligonucleotides for one given genomic position.

FIG. 6B schematically shows total DNA (a; squiggly line); b, c, d and e represent different sheared DNA fragments from total DNA and representing the genomic equivalents of the oligonucleotides shown as in FIG. 6A, together with the flanking sequence (squiggles). The flanking sequence includes internal mobile element (ME) sequence shown as a hatched box.

FIG. 6C schematically shows oligonucleotides on a solid support, as in FIG. 3A, hybridized to genomic DNA. In this particular example, only the empty site [left flank/right flank (FL/FR)] oligonucleotide matches the genomic DNA completely (a). For oligonucleotide comprising left flank/mobile element (FL/ME), only the mobile element (ME) matches for one particular sheared fragment of genomic DNA (b); for mobile element/right flank (ME/FR), only the right flank (FR) segment matches in another case (c). In other cases different patterns would be detected.

FIG. 6D schematically depicts the detection step carried out. A labelled dideoxynucleotide is added which can be incorporated at the end of the oligonucleotide providing the oligonucleotide is hybridized to genomic DNA as template. The nucleotide sequence at the genomic position adjacent to the region matching the oligonucleotide is known, and therefore the particular nucleotide, which will be incorporated (A, C, G, T or U) is known. In the example shown, oligonucleotides b and c are not extended because they lack the hybridized genomic DNA.

FIG. 6E schematically shows the scoring of the detection reaction. The scoring is shown schematically. Oligonucleotide left flank/right flank (FL/FR) (a) represents the empty site, and gives a positive signal. Oligonucleotides left flank/mobile element (FL/ME) (b) and mobile element/right flank (ME/FR) (c) represent the full site, and both give no signal. Hence, the site is confirmed as empty.

FIG. 7 shows the 1767 nt sequence of sb17.seq (SEQ ID NO:20). Using primer 7286 (SEQ ID NO:18) and (CTC)₉C (SEQ ID NO:19) in Retrotransposon Microsatellite Amplified Polymorphism (REMAP) method, a polymorphic band was identified that was present only in spring barley accessions but not in winter barley accessions. The band was excised from the ethidium bromide stained agarose gel, cloned, and sequenced. The LTR of the BARE-1 insertion is underlined. It represents the end of an LTR inverted with respect to the sense direction of the open reading frame. The predicted 5 bp direct repeat generated by the insertion, CCACT, is in bold italics.

FIG. 8 shows the 3186 nt sequence of wb17.seq (SEQ ID NO: 21). Using primer 7286 (SEQ ID NO: 18) and (CTC)₉C (SEQ ID NO: 19) in the Retrotransposon Microsatellite Amplified Polymorphism (REMAP) method, a polymorphic band was identified that is present in winter barley accession. The band was excised from the ethidium bromide stained agarose gel, cloned, and sequenced. Winter barley lacks the BARE-1 insertion at this genomic position. This sequence is almost completely identical with the sb17 sequence (SEQ ID NO: 20) except that it lacks the BARE-1 sequence. The insertion site of the BARE-1 in sb17 (SEQ ID NO: 20) is marked with an arrow in the wb17 (SEQ ID NO: 21) sequence, and is located between nucleotides 1511 and 1512 in wb17 (SEQ ID NO:21). These flanking nucleotides are underlined. The 5 bp sequence which forms the direct repeat in sb17 (SEQ ID NO: 20) is in bold.

FIG. 9 shows how the mobile element/left flank (ME/FL) flanking region can be predicted from the BARE-1 LTR, the predicted 5 bp direct repeat, and wb17 sequences respectively.

FIG. 9A shows the sequence representing the first 100 nt of the inverse-orientation BARE-1 LTR and c.a. 100 nt predicted for the sb17 5′ joint region (SEQ ID NO: 23).

FIG. 9B shows the left and right flanks (FL and RF) and the inserted mobile element (ME) in summary in the following manner: left flank (FL)/mobile element (ME) (SEQ ID NO: 24); right flank (FR)/mobile element (ME) (SEQ ID NO: 25). These oligonucleotides are synthesized, optionally end-protected, and attached to the chip and represent a single genomic position for maize. Oligonucleotides for an additional 50 or more genomic positions are derived and treated in a similar manner.

### DETAILED DESCRIPTION OF THE INVENTION

### Abbreviations

AFLP Amplified Fragment Length Polymorphism

BARE Barley Retrotransposon

FL left flanking region; left flank

FR right flanking region; right flank

IRAP Inter-Retrotransposon Amplified Polymorphism

LINE Long Interspersed Element

LTR Long Terminal Repeat

MAS Marker Assisted Selection

ME Mobile Element

MITE Miniature Inverted Repeat Transposable Element

RAPD Randomly Amplified Polymorphic DNA

REMAP Retrotransposon Microsatellite Amplified Polymorphism

RBIP Retrotransposon-based Insertion Polymorphism

RFLPRestriction Fragment Length Polymorphism

SINE Short Interspersed Element

SNP Single Nucleotide Polymorphism

SSR Simple Sequence Repeat

SSAPSequence Specific Amplified Polymorphism

STR Short Tandem Repeat

TRIM Terminal-Repeat Retrotransposon In Miniature

VNTR Variable Number of Tandem Repeat

### Terms Used in the Disclosure

In the present disclosure most of the terms used have the same meaning as they generally have in the fields of genetics, human medical diagnostics, recombinant DNA techniques, molecular biology and in plant and animal breeding. Some terms are, however, used in a somewhat different way and are explained in more detail below.

The term “genetic identity” means genetic diversity, genomic variation or polymorphism, allelic variation or genetic uniqueness of an individual within a defined population pool characterized by genomic variation or polymorphism, allelic variation representing genetic diversity. The population pool includes plants, especially crop plants, including barley, potato, brassica, etc., animals, especially animals in farming including cows and horses etc., or pet animals, including dogs, cats, etc., without excluding human beings.

The term “polymorphism” means a quality or characteristic feature occurring in several different forms. For example in the present disclosure the difference(s) i.e. “polymorphism(s)” between the hybridization patterns, means that a mobile element (ME) is present or absent at a particular site or adjacent to a particular flanking sequence in the defined population pool.

The term “co-dominant” means that e.g. in a diploid organism, heterozygous and homozygous alleles can be distinguished from each other. The markers produced by the present invention are co-dominant markers.

In the present disclosure the term “mobile element (ME)” means genetic element(s), which are interspersed throughout the genomes of higher plants and animals as well as prokaryotes (Lodish, et al., Molecular Cell Biology, W. H. Freeman and Company, NY, 2000). They range from tens or hundreds to a few thousands of base pairs in length and can be copied and reinserted into a new site in the genome by transposition (retrotransposon-like mobile elements) or they can excise themselves and reinsert elsewhere in the genome, either autonomously, or non-autonomously (transposons).

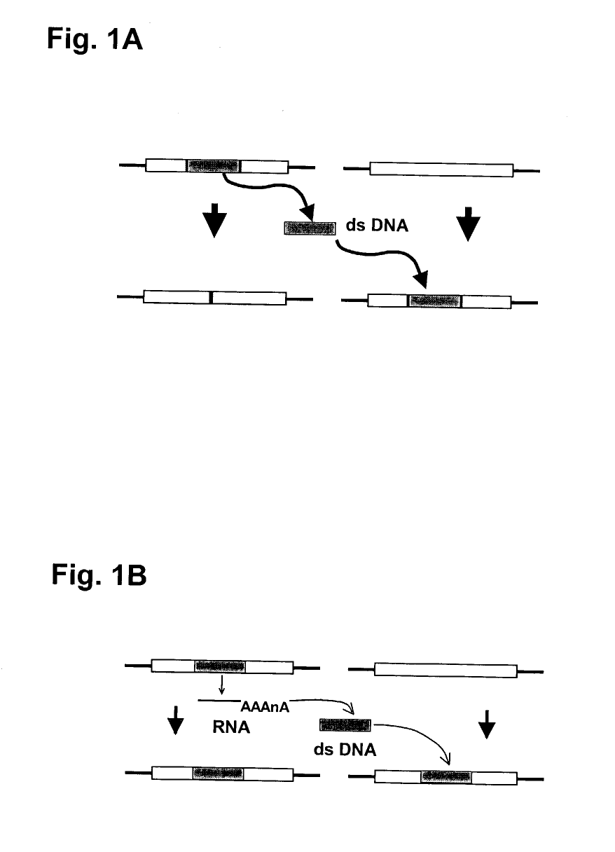

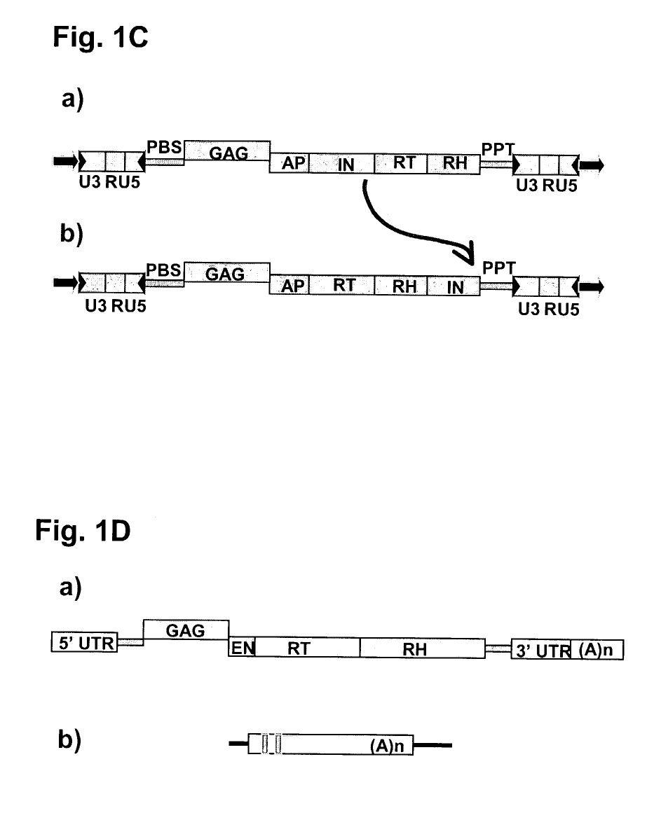

Mobile elements (MEs) can be divided into two categories: 1) DNA-mediated transposons (FIG. 1A), which transpose directly as DNA and are generally referred to as transposons. DNA transposons include bacterial insertion sequences (IS elements, e.g. IS1, IS10), bacterial transposons (e.g. Tn9) and eukaryotic transposons (e.g. P element from Drosophila, Ac and Ds elements from maize), 2) RNA-mediated transposable elements (FIG. 1B). Said elements transpose via an RNA intermediate transcribed from the mobile element by an RNA polymerase. Thereafter they are converted back into double-stranded DNA by a reverse transcriptase. They are called retrotransposons, because their movement is analogous to the infection process of retroviruses. Retrotransposons include virus-like retrotransposons, such as Long Terminal Repeat (LTR) retrotransposons (FIG. 1C) (e.g. Ty element from yeast, copia-like and gypsy-like elements) and non-virus-like retrotransposons, such as non-LTR-retrotransposons (FIG. 1D) [e.g. F and G elements (Drosophila), Long Interspersed Elements (LINEs) and Short Interspersed Elements (SINEs) (mammals and plants), (Alu) sequences (humans)]. Non-autonomous retrotransposons include also Terminal-Repeat Retrotransposons In Miniature (TRIM) elements (Witte et al., Proc Natl Acad Sci 98:13778-13783, 2001). Retrotransposons do not excise as do DNA transposons, but instead they duplicate themselves and reinsert their duplicated copies elsewhere in the genome. Retrotransposons are, therefore, implicated in the evolution of the genome since the essentially random insertion of duplicated sister copies into the genome will change the overall organization of the genome. Retrotransposons have been widely used to study the pedigree of breeding populations because in each generation there is a certain probability that a new and characteristic retrotransposon profile will be produced.

Mobile element (ME) insertions of retrotransposons or DNA transposons generate insertions comprising hundreds to thousands of base pairs in the genome (FIG. 1). These are polymorphic when the mobilization event has occurred before the last common ancestor of the two genotypes are compared. Two independent insertions occur quite rarely for most mobile elements (MEs) at precisely the same location in two genomes, given greater than 10⁹ bp in an average eukaryotic genome.

The term “sample DNA” means polynucleotides representing the total DNA of the sample, which is used in unlabeled and optionally fragmented form and which is rendered single-stranded before use. The sample DNA may originate from any specimen or organism, e.g. from plants, animals, human beings, bacteria, fungi or it can be ancient DNA.

The term “oligonucleotide” means any polymer of single nucleotides, which is used in the present invention attached in single stranded form to the solid support in order to demonstrate genetic identity in a DNA sample from any specimen. The term “oligonucleotide” is not restricted to any specific number of nucleotides. In other words the term “oligonucleotide” means a polymer typically made up of approximately 20 nucleotides and the upper limit is any length that can be synthesized using an oligonucleotide synthesizer. The current upper limit is about 150 bp. Naturally; it can be higher if the capacity of the oligonucleotide synthesizer is improved. Even if the Figures show only one oligonucleotide the term oligonucleotide and especially the term oligonucleotides or oligonucleotide sequences means a multitude of substantially identical oligonucleotides.

The term “labelled oligonucleotides” means labelled polynucleotides that fully correspond to the attached oligonucleotide sequences.

The “oligonucleotides” are single-stranded polynucleotide sequences. Each oligonucleotide comprises two different parts of varying lengths, one part being the region flanking a mobile element (ME) and the other comprising the terminal end of said mobile element (ME) or the flanking region situated on the opposite side of the first flanking region. The oligonucleotide sequences have a size of approximately 20 nucleotides, more preferably at least 25, most preferably more than 30 in order to provide a sufficiently stable hybridization product between the attached oligonucleotide and the sample DNA. The oligonucleotides comprise three alternatives for each mobile element (ME), the left flanking region (FL) combined with one terminal end of the mobile element (ME), the right flanking (FR) region combined with another terminal end of the mobile element (ME) or a combination of the left and right flanking regions (FL+FR) for detecting the absence of a mobile element (ME). The oligonucleotide representing the flanking region may comprise regions flanking the flanking region to enable more stable hybridization and better resolution.

The term “set of oligonucleotides” means a number of polynucleotide sequences capable of recognizing the presence or absence of specific defined mobile elements (MEs) or specific genomic positions. Several sets of oligonucleotides, at least one for each available mobile element (ME) or genomic position can be used. Alternatively, one mobile element (ME) may be combined with different flanking regions or one flanking region may be combined with different mobile elements (MEs). An estimated minimum of sets of oligonucleotides for obtaining an optimal mapping or fingerprinting result, for example, for breeding purposes is at least 70 for a diploid organism having 7 chromosomes. This, however, does not provide an obstacle for using the method and the test kit of the present invention with a smaller or larger number of sets of oligonucleotides. The “set of oligonucleotides” may comprise a single oligonucleotide detecting the presence of the mobile element (ME) in an integration site. Alternatively, the set may comprise an oligonucleotide detecting the mobile element (ME) in combination with another oligonucleotide detecting the lack of the mobile element (ME). Said two types of oligonucleotides, forming a set of paired oligonucleotides, which are capable of identifying both a full and an empty integration site simultaneously. The “set of oligonucleotides” may also comprise three or more parallel oligonucleotides representing the same integration site, i.e. the three oligonucleotides detect both the left and the right terminal ends of the mobile element (ME) and the lacking mobile element (ME). Additional oligonucleotides for the set are obtained when the complementary strands are used as well. Said parallel set of oligonucleotides provides a more reliable result, by confirming that both ends of the mobile element (ME) are present. The “set of oligonucleotides” may be attached to a single solid support or solid support comprising of one or more separate solid supports. The “one set of oligonucleotides” is a single oligonucleotide, a pair of oligonucleotides or parallel oligonucleotides representing a mobile element (ME).

The term “scoring” means comparing the recordable hybridization pattern which can be recorded and wherein the presence or absence of hybridization demonstrates the presence or absence of the corresponding mobile element (ME) insertion, respectively. The scored results are collected and assessed either as full, empty, failure or null alleles.

The term “full site” means a mobile element (ME)-containing form of a genomic position or integration site. It can be demonstrated with an oligonucleotide sequence attached to the solid support, which comprises a terminal end of the mobile element (ME) with respective flanking sequences. The DNA sequence from mobile element (ME)-containing genomic position hybridizes with the oligonucleotide attached to the solid support composed of two distinct sequence regions of varying lengths, one distinct sequence region being composed of the flanking region of a mobile element (ME) and the other of the terminal end of the mobile element (ME) and the other part comprising the terminal fragment of the mobile element (ME), but not to the oligonucleotide composed of two opposite flanking regions.

The term “empty site” means a mobile element (ME)-absent form of a genomic position or integration site, which can be demonstrated with an oligonucleotide sequence attached to the solid support corresponding to an empty site or genomic position in which the mobile element (ME) is absent or lacking. The DNA sequence from the genomic position lacking the mobile element (ME) therefore hybridizes with the oligonucleotide sequence or sequences, attached to the solid support composed of two parts of equal or varying lengths, one of which comprises the left flanking region (FL) and the other the right flanking region (FR) lacking a mobile element (ME).

The term “failure allele” corresponds to the loss of the genomic position, or more precisely to the loss of the ability to hybridize to the genomic position, and is the score given when both the empty oligonucleotide(s) and the full oligonucleotide(s) give a no-hybridization response. The term “failure allele” means an allele that will score as full with an left flank/right flank (FL/FR) oligonucleotide but empty with a left flank/mobile element (FL/ME) or right flank/mobile element (FR/ME) oligonucleotide. “Failure allele” could result from any of a series of causes, such as accumulation of sufficient insertion/deletion point mutations in the flank destroying the ability to hybridize, low-quality probe, contaminations effecting hybridization efficiency or detection, etc.

The term “null allele” is a subset of “failure allele” and corresponds to the loss of the genomic position. “Null allele” means that the site itself comprising the left flank (FL), right flank (FR) and possibly the mobile element (ME) is absent from the genome so that the site will score as full with a left flank/right flank (FL/FR) oligonucleotide but empty with a left flank/mobile element (FL/ME) oligonucleotide or right flank/mobile element (FR/ME) oligonucleotide. “Null allele” would mean specifically that the flanks are missing due e.g. to a recombination event.

The term “a region flanking a mobile element (ME)” means the region immediately flanking the mobile element (ME), which may include tandem repeats, other mobile elements (MEs), genes, promoters, introns, exons, etc., but may also include other contiguous regions flanking the flanking region.

The term “a terminal end of the mobile element (ME)” means either the 5′- or 3′-terminal end of the mobile element (ME) or their complementary strands or in any combinations thereof.

The term “the flanking region situated on the other side of the first flanking region” means that the mobile element (ME) is surrounded by two flanking sequences one on each side of the integration site.

The term “solid support” means a solid non-aqueous matrix and may be a membrane, filter, slide, plate, chip, dish, composed of a material selected from a group consisting of glass, plastics, nitrocellulose, silicons, etc. Preferred solid supports are membranes, filters, slides, plates, dishes, and microwell plates. The “solid support” can be composed of a material selected from a group consisting of glass, plastics, nitrocellulose, nylon, polyacrylic acids, silicons, etc. The solid support together with the oligonucleotides attached to it form the test kit or product of the present disclosure.

The term “recording of the hybridization state” means any method by which the hybridization may be detected and includes any method by which the hybridization is made visible or otherwise detectable, but the term also includes methods, which require a given analytical instrument to achieve detection or permit the hybridization state to be recorded for automated applications of the method.

The term “recording” means measuring or detecting the presence or absence of hybridization for each pair of oligonucleotide sequences using any labels and method allowing the recording of the hybridization state.

The term “hybridization” refers to the process of bringing two complementary strands of a nucleic acid. i.e. two separate DNA polynucleotide, oligonucleotide strands, or one DNA and one RNA strand together by hydrogen bonding. Hybridization is generally performed in a suitable buffer, such as but not limited to 6×SSC, 0.05% sodium pyrophosphate, 0.1% SDS, as defined in common laboratory practice (Ausubel, et al., 2001, John Wiley & Sons, Inc., New York, vol. 1, unit 6.4.2. supplement 13), at a suitable temperature such as, but not limited to 53° C., generally 12° C. below the determined melting temperature of the hybrid in view of the salt concentration in the hybridization buffer.

The term “post-hybridization treatments” means removal of single-stranded sample DNA which is not fully hybridized to the oligonucleotide sequence(s) attached to the solid support by applying washing steps at different stringencies or removal of partly hybridized single strands protruding from the oligonucleotide(s) by optional digestion treatments or enzyme treatment with nucleases specific to single-stranded nucleotide sequences. Washing stringencies follow common laboratory practice (Ausubel, et al., 2001, John Wiley & Sons, Inc., New York), but will generally be about 60° C. in a buffer containing 6×SSC though it can be higher or lower. Generally, washing is carried out at below the melting temperature of the hybridized molecule.

The term “a recordable label” means any labels or markers, which may be used to indicate or trace that hybridization has occurred. They may be visible or detectable labels, which may be recordable as such or which can be made detectable or recordable when contacted with other reagents. The labels or markers which are recordable by their electrochemical or magnetic properties, fluorescence, luminescence, their infra-red absorption, radioactivity or by enzymatic reactions are especially appropriate, but any tracer tags, which are easily recordable by automatic means or instruments can be used.

Preferred recordable labels are fluorochromes or fluorophors, such fluorescent labels may be found among thiol-reactive fluorescent dyes, such as 5-(2-((iodoacetyl) amino)ethyl)aminonapthylene-1-sulfonic acid) (1,5- IEDANS) or fluorescein, Bodipy, FTC, Texas Red, phycoerythrin, rhodamine, carboxytetramethylrhodamine, DAPI, an indopyras dye, Cascade Blue, Oregon Green, eosin, erythrosin, pyridyloxazole, benzoxadiazole, aminonapthalene, pyrene, maleimide, coumarin, Lucifer Yellow, Propidium iodide, porhyrin, CY3, CY5, CY9, lanthanide cryptate, lanthanide chelate, or derivatives or analogues of said tracer molecules. The fluorescent labelled oligonucleotides are especially useful in automated or semi-automated recording.

The term “shearing of sample DNA” means any chemical, mechanical or physical means by which the long DNA strands may be fragmented in order to obtain the mobile elements (ME) on separate DNA fragments for recording. Methods for fragmenting DNA include restriction enzyme treatments, sonication, etc.

The term “end-protection” means that the attached oligonucleotides are protected in order to stabilize the test kit with the solid support and to avoid the oligonucleotides from being damaged thus preventing a false or artifactual score from being recorded. Useful end-protection is obtained with known methods selected from a group consisting of 5′OH derivatization, amino-derivatization, etc.

The term “test kit” means the solid support with one or more sets of optionally paired or parallel oligonucleotides attached thereto. The paired or parallel oligonucleotides mean different oligonucleotides representing the same mobile element (ME). It is self-evident that each set contains a multitude of substantially identical oligonucleotides. The test kit may optionally be provided in a packaged combination with auxiliary reagents and instructions.

The term “half hybrid” means the probe-sample DNA hybrid which is only partially double-stranded, because the sample DNA is incompletely homologous to the probe oligonucleotide. The term “full hybrid” means the probe-sample DNA hybrid, which is fully double-stranded. A discriminatory hybridization temperature allows the full-length hybrid, “full hybrid” to remain annealed while the half-length hybrid, “half hybrid” would melt. The key of the present method is distinguishing between two states, “half hybrid” and “full hybrid”. These two states correspond to situation when a probe for a full insertion site left flank/mobile element (FL/ME) for a mobile element is hybridized to sample DNA containing only empty site fragments left flank/right flank (FL/FR); in this case the left flank (FL) segment would hybridize but the mobile element (ME) portion of the probe would not be covered by a region of the probe DNA contiguous with left flank (FL).

The main objectives of the present invention are to provide a reliable method and a test kit useful for genetic identity determination, phylogenetic studies, parenthood determinations, genotyping, haplotyping, pedigree analysis, forensic identification, human medical diagnostics and/or plant or animal breeding particularly with co-dominant scoring. In the demonstration of genetic diversity a reliable method and test kit should exploit defined and conserved DNA entities in the genome, and allow scoring of changes which are spread throughout the genome at high frequency and thereby enable, for example, dense and well distributed recombination maps to be generated.

The method and the test kit of the present disclosure apply molecular markers or entities, which are heritable as simple Mendelian traits and are easily scorable. The markers allow detailed studies of inheritance and variability, the construction of linkage maps, and the diagnosis of individuals or lines carrying certain linked genes. Phenotypic and biochemical (enzyme) markers, which have previously been used, tend to have the disadvantages of a low degree of polymorphism limiting their mapability in crosses, relatively few genomic positions, limiting the density of maps which can be produced, and environmentally variable expression, complicating scoring and the determination of genotype. These have been superseded by DNA-based methods, which generate fingerprints or molecular markers, which are distinctive patterns of DNA fragments resolved, for example, by electrophoresis in agarose or acryl amide gels and detected by staining or labelling. A molecular marker is in essence a nucleotide sequence corresponding to a particular physical location in the genome. Its occurrence or size should be polymorphic, that is varying sufficiently, to allow its pattern of inheritance to be followed.

The general principle of the present invention is to provide a method and a test kit, which applies a solid support or chip containing permanently or non-permanently attached scorable oligonucleotide sequences, which are capable of recognizing particular genomic positions in the genome. Principally, any domain in the genome that has a length feasible for hybridization and screening could be scored. Polymorphisms within these sites could be scored if the hybridization would be followed by digestion with an endonuclease. The endonuclease would cleave mismatches or bubbles within the hybridized sample/oligonucleotide pair. The resulting fragmentation could then destabilize the hybrid and allow release of those cleaved fragments.

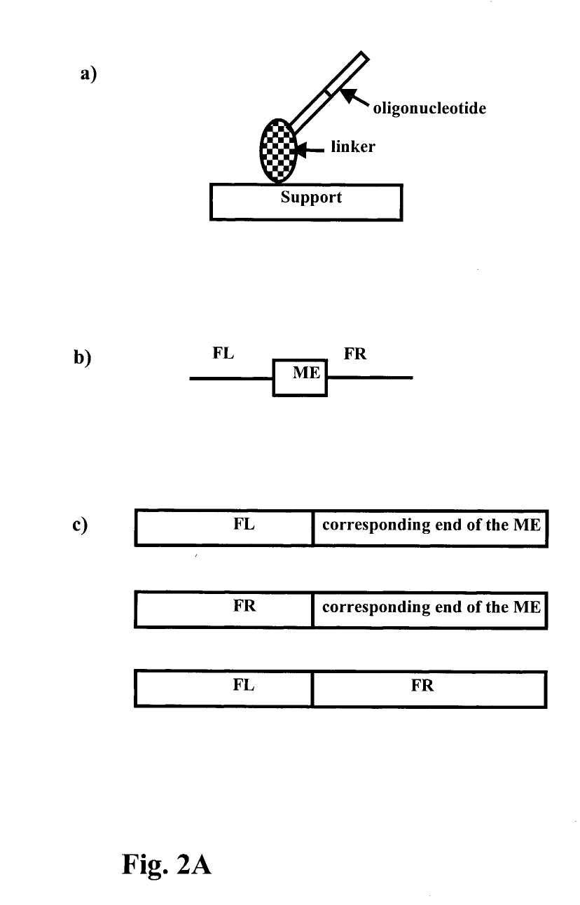

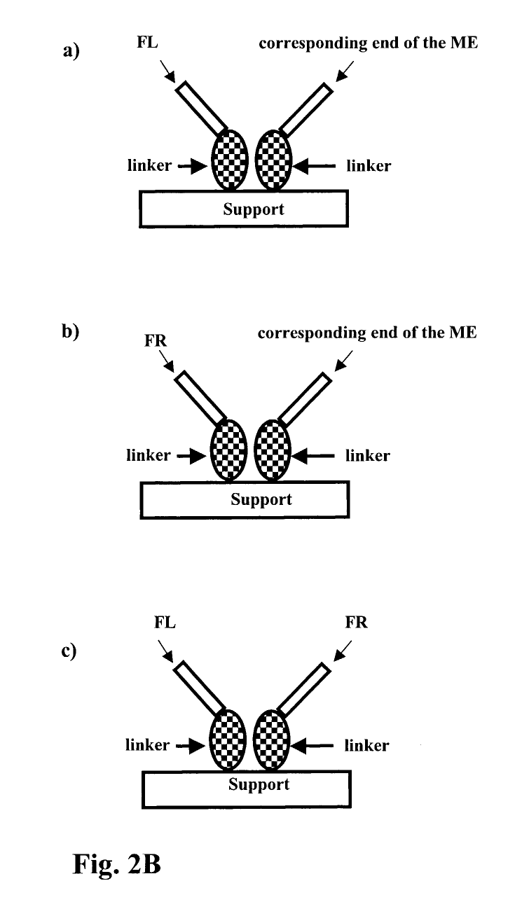

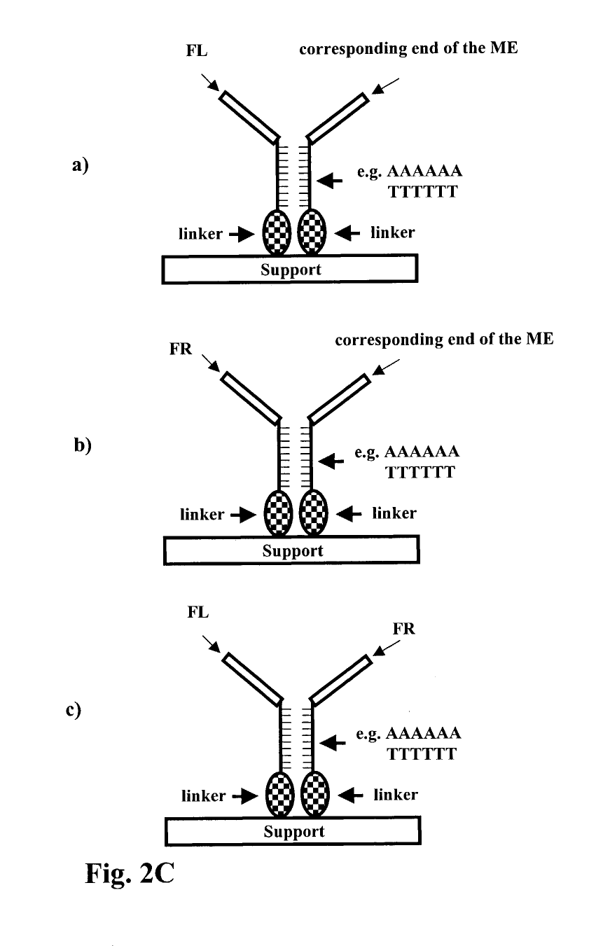

More specifically, the oligonucleotide sequences for each genomic position may be present as three substantially different types of oligonucleotides, i.e. they may comprise the left flanking region (FL) combined with one of the terminal ends of a mobile element (ME), the right flanking region (FR) combined with another terminal end of the mobile element (ME) or a combination of the left and right flanking regions (FL+FR) surrounding the integration site (FIG. 2A). Said oligonucleotides can be used one by one, as pairs or in parallel, or combining all three oligonucleotide types. Preferably, each genomic position is represented by a certain defined flanking region combined with a certain defined mobile element (ME), but because the same mobile element (ME) can be inserted in different integration sites, it is also possible to combine each defined mobile element (ME) with different kinds of flanking regions.

The oligonucleotide sequences used in the present invention comprise approximately 20 nucleotides, more preferably at least 25, most preferably more than 30 nucleotides in order to provide a sufficiently stable hybridization product between an attached oligonucleotide(s) and sample DNA. It is to be noted that the oligonucleotides are composed of two distinct sequence regions and therefore the oligonucleotides should be sufficiently long to enable hybridization both with the flanking region and the mobile element (ME), or with each of the flanking regions on both sides of the integration site of said mobile element (ME) when the mobile element (ME) is lacking. In certain embodiments of the present invention the part of the oligonucleotide arrangement representing the flanking region may comprise regions flanking the flanking region to enable more stable hybridization and better resolution. Therefore, the length of the flanking region derived part of the oligonucleotide -may be greater than that representing the terminus of the mobile element (ME). The length of each oligonucleotide is determined by the fact that it should allow a sufficiently stable hybridization product between the attached oligonucleotide and the sample DNA. Naturally, both parts can be equally long.

Typically, more than one set of oligonucleotides, each capable of recognizing the presence or absence of a specific and defined mobile element (ME) or genomic position, is used. More than one oligonucleotide pair per homologue of the subject to be identified preferably should be used. By way of example, for a diploid organism with seven chromosome pairs it can be calculated that for obtaining an optimal mapping result at least 70-80 sets of oligonucleotide pairs, each representing a certain genomic position or mobile element (ME), are required. For organisms with more chromosomes more oligonucleotides are desirable. The lower limit is one oligonucleotide pair and the upper limit is set by the desired resolution capacity of the method and the test kit.

Hybridization is preferably recorded in situ by any conventional labelling system, applying for instance terminal transferase and conventional recordable labels. As an alternative to in situ labelling the hybridized sample DNA may be released from the solid support and subsequently hybridized with labelled polynucleotide sequences corresponding to each of the original oligonucleotide sequences attached to the solid support. Hybridization is optionally reversible and the solid support can be returned to its original state for reuse.

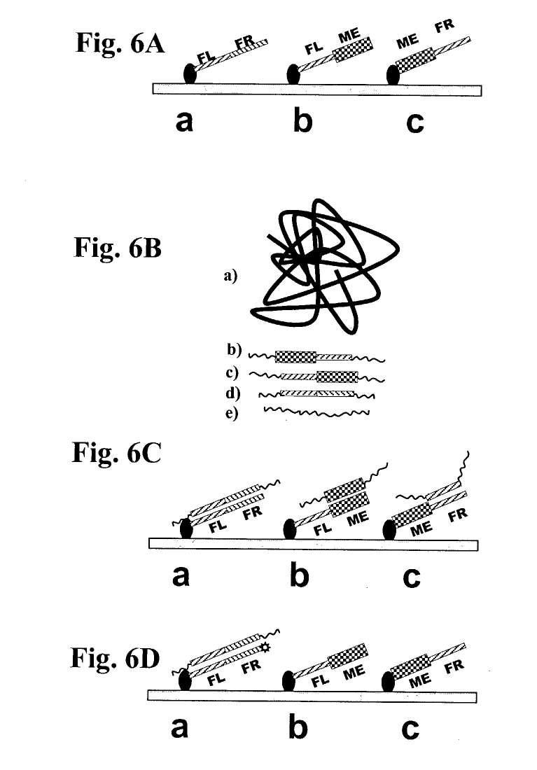

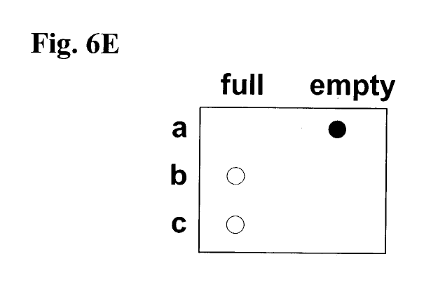

A labelled dideoxynucleotide can be incorporated at the end of the oligonucleotide provided that the oligonucleotide is hybridized to genomic DNA as template. The nucleotide sequence at the genomic position adjacent to the region matching the oligonucleotide is known and therefore the particular nucleotide which will be incorporated (A, C, G, T or U) is known (FIG. 6).

Co-dominant scoring is achieved using paired, i.e. two or parallel, i.e. three, flanking oligonucleotide sequences. The results obtained are recorded as full, empty, failure or null alleles and can be used to distinguish between heterozygous and/or homozygous genotypes. In regards the use of the method for marker assisted selection (MAS), the number of informative flanking sequence DNA pairs depends on where the sequence pairs map relative to known genes of interest.

Optional post-hybridization treatments, including washing and digestion, are provided in order to remove sample DNA not fully hybridized to the solid support-attached oligonucleotide sequences, for example before and after labelling. The presence or absence of hybridization is recorded using any method allowing the recording of the hybridization state.

The present invention discloses a technique which uses sets of oligonucleotide sequence attached to a solid support, one part of each oligonucleotide sequence comprising a region flanking a mobile element (ME) and the other part of said oligonucleotide sequence comprising a terminus of a mobile element (ME), respectively or the opposite flanking site if the mobile element (ME) is lacking.

Accordingly, an objective of the present invention is to provide a method for detecting genomic variations based on insertion of mobile elements (MEs) present in any given position in a pool of genotypes using a solid support with permanently or non-permanently attached oligonucleotides. The method allows identification of genomic positions containing a mobile element (ME) or lacking a mobile element (ME). The objective is to provide a desired level of resolution within a defined population pool with a great diversity of genotypes. The method allows co-dominant scoring, i.e. distinguishing between heterozygous and homozygous genotypes. The invention further relates to a test kit comprising one or more means for detecting genomic variations based on insertions of mobile elements (MEs).

A mobile element (ME) can be any mobile genetic element of a type including DNA transposons such as eukaryotic transposons, bacterial insertion sequences and bacterial transposons, retrotransposon including virus-like retrotransposons such as Long Terminal Repeat (LTR) retrotransposons e.g. gypsy-like and copia-like elements (Kumar and Bennetzen, Annu. Rev. Genet. 33:479-532, 1999) especially from barley (BARE-1, BARE-2, BARE-3, Sukkula, Sabrina, Nikita, BAGY-1, BAGY-2, etc.), non-virus-like retrotransposons such as non-LTR retrotransposons [e.g. Long Interspersed Elements (LINEs) and Short Interspersed Elements (SINEs) in mammals and (Alu) sequences in humans], bacteriophages, etc., non-autonomous elements including Miniature Inverted Repeat Transposable Elements (MITEs) (Wessler, et al., Curr. Opin. Genet. Dev. 5: 814-821, 1995), which are highly-deleted versions of mobile elements (MEs), or Terminal-Repeat Retrotransposons In Miniature (TRIM) (Witte, et al., Proc. Natl. Acad. Sci USA 98:13778-13783, 2001).

In preferred embodiments of the present invention retrotransposons, which recently have been developed as molecular marker systems meeting many of the requirements for an ideal marker system, are used. Retrotransposons are preferred because their replicative means of transpositions gives increased stability of the genomic position states and thereby more powerful phylogenetic resolution, compared with DNA transposons which may be mobilized out of a site.

Accordingly, the departure point of the present method is the concept that rather than placing single-stranded oligonucleotides representing unlabeled total sample DNA on a solid support, which can be made of any material, the solid support carries more than one set of permanently attached, unlabeled, sequence-defined oligonucleotides, representing, for example, mobile elements (MEs) and their insertion site junctions.

The method of the present invention allows unlabeled, optionally fragmented, total DNA of the sample to hybridize with more than one set of oligonucleotide sequences attached to the solid support, each oligonucleotide sequence being composed of two parts of varying length, one part comprising a region flanking a mobile element (ME) and the other part comprising a terminal end of the mobile element (ME) or the flanking region situated on the opposite side of the first flanking region.

Attached to the solid support is such a number of oligonucleotides corresponding to integration sites of mobile element (ME), i.e. insertion sites established to be polymorphic within a potential pool of genotypes to be typed, that meaningful mapping or fingerprinting and a desired level of resolution between genotypes is obtained. In practice, this means that at least one, preferably more sets of oligonucleotides has to be identified for each homologue to be scored in the organism. In some cases, even a single polymorphic site can serve to resolve a basic division between classes of genotypes. Such cases include for example distinguishing between spring and winter barleys, between strains of bacterial or fungal pathogens, or between human populations.

Each of said sets of optionally paired or parallel oligonucleotide sequences comprise one oligonucleotide sequence corresponding to a full site and the other to an empty site. The full site comprises an oligonucleotide sequence being composed of two contiguous parts, one of which comprises the flanking region of a mobile element (ME) and the other comprises one terminal end of the mobile element (ME). The oligonucleotide sequence corresponding to the empty site comprises an oligonucleotide sequence composed of two parts of equal or varying lengths one of which is the left flanking region (FL) and the other the right flanking region (FR) surrounding a site lacking a mobile element (ME). The left flanking region (FL) is for example combined with the 5′ end of the mobile element (ME) and the right flanking region (FR) is for example combined with the 3′ end of the mobile element (ME) or vice versa. The oligonucleotides can be prepared from both strands and they can be used in any combination. They are combined with an oligonucleotide recognizing an empty site comprising the left and right flanking regions (FL+FR).

More specifically each oligonucleotide sequence is composed of two parts of equal or varying length, one part comprising a region flanking a mobile element (ME) and the other part comprising a terminal end of the mobile element (ME), or the flanking region situated on the other side of the first flanking region. In an alternative embodiment the sequence representing the flanking region is longer than the part representing the mobile element (ME).

Principally, three different types of oligonucleotides in each set of oligonucleotides representing a mobile element (ME) can be used in the present invention (FIG. 2A). An oligonucleotide representing the flanking region and the terminal end of the mobile element (ME) can be designed as one single contiguous sequence, which is attached by a linker to the solid support and its pair comprising the two flanking regions surrounding the integration site and representing an empty site is also designed as one single contiguous sequence which is attached by a separate linker to the solid support.

In an alternative embodiment the flanking region and the mobile element (ME) of the oligonucleotide can be placed separately on two separate linkers, which are attached to the solid support in close proximity to each others (FIG. 2B). The corresponding pair comprising the flanking regions surrounding the integration site is- also attached by additional linkers to the solid support.

Each part of the oligonucleotides described above can be provided with a synthetically prepared elongated sequence, a so-called stem sequence (FIG. 2C). The stem sequence is a region that is complementary to a similar region on another oligonucleotide for the purpose of annealing the two oligonucleotides together. Therefore the stem serves to position the two oligonucleotides so that both of the oligonucleotides together hybridize with the DNA sequence for the genomic position. Said partially complementary oligonucleotides are attached to the solid support through a linker(s) attached to the double stranded end.

Suitable nucleotide sequences useful for constructing synthetic oligonucleotide sequences for manufacturing the test kit can also be obtained, e.g. by screening bacterial artificial chromosome (BAC) libraries and sequencing regions containing mobile elements (MEs) or by the Sequence-Specific Amplified Polymorphism (SSAP) method to get PCR-products which define the insertion site of the mobile element (ME) in a given genome. The basic strategy is to identify the flanking sequence on each side of a retrotransposon either by use of a standard PCR procedure termed inverse-PCR or by the standard method called genome walking (Siebert, et al., Nucl. Acids Res. 23: 1087-1088, 1995) and then to use the unique flanking DNA sequence to develop the markers.

The regions flanking mobile elements (MEs) at known genomic positions are used as primers in combination with primers to the mobile element (ME) and amplification is carried out by PCR methods. The resulting PCR products can be isolated and the corresponding sequences characterized. Subsequently, the said new mobile elements (MEs) can be used to identify new flanking regions useful for designing flanking region PCR primers for use in the test kit. When a sufficient number of useful mobile elements (MEs) and flanking regions have been identified, they can be used as models for producing oligonucleotide sequences useful for manufacturing the test kit.

The oligonucleotides, which can be produced by recombinant DNA techniques or synthetically or semi-synthetically, may be attached to the solid support by a variety of means. The oligonucleotides should not be sterically constrained so as to interfere with hybridization. The oligonucleotide sequences are optionally end-protected. The end-protection of the oligonucleotide is carried out by per se known methods selected from a group consisting of e.g. 5′OH derivatization and amino-derivatization.

Unlabeled, optionally fragmented total DNA of the sample may originate from any specimen, from any species and/or from any organism, e.g. from plant, animal, human, bacteria, fungi and/or ancient DNA. Optionally, DNA representing total DNA is sheared to fragments of approximately ca. 500 bp or less with physical, mechanical or enzymatic means e.g. enzymatic digestion with a frequent cutter or preferably by sonication. The purpose of shearing is to physically separate the particular genomic positions to be scored onto different pieces of DNA to increase the efficiency of the process. The sample DNA is dissociated to a single-stranded state by per se known methods e.g. by boiling in a buffer similar to that used for other types of hybridization.

The solid support comprises a membrane, filter, slide, plate, chip, dish, composed of a material selected from a group consisting of glass, plastics, nitrocellulose, nylon, or mixed compositions or hybrid media.

The hybridization reaction takes place under conditions that allow the optionally fragmented single-stranded sample DNA to anneal to the oligonucleotide sequences attached to the solid support in its full length.

Hybridization is generally performed in a suitable buffer such as but not limited to 6×SSC, 0.05% sodium pyrophosphate, 0.1% SDS, as defined in common laboratory practice (Ausubel, et al., 2001 John Wiley Sons, Inc. New York, vol.1., unit 6.4.2. Supplement 13) at a suitable temperature such as, but not limited to 53° C., generally, at 12° C. below the determined melting point of the hybrid in view of the salt concentration in the hybridization buffer. Optionally, buffers such as 1×PCR buffer (50 mM KCl, 10 mM Tris-HCl pH 9 (at 25° C.), 0.1% Triton-X 100, 1.5 mM MgCl₂) can be used.

Following hybridization optional post-hybridization treatments are carried out in conditions, including salt concentration and temperature, releasing all sample DNA not completely or almost completely hybridized to oligonucleotide sequences attached to the solid support. Optional post-hybridization treatments include removal of single stranded sample DNA which is not fully hybridized to the oligonucleotide sequences attached to the solid support with a washing step at different stringencies and optional digestion treatments to remove single stranded sample DNA fragments not fully corresponding to the attached oligonucleotide sequences. Washing is generally carried out by the well-known procedure of incubating in a buffer consisting of, but not limited to, 6×SSC, 0.05% sodium pyrophosphate at 65° C., or at just above the calculated melting temperature of the hybrid.

In one embodiment of the invention, the hybridized genomic fragments, which are mostly considerably longer than the oligonucleotides, are trimmed by addition of a digestion step following the hybridization. In the digestion step the unhybridized oligonucleotides or partly hybridized single-stranded oligonucleotides are removed by enzymatic digestion with an enzyme such as a single-strand-specific nuclease, preferably an exonuclease, leaving the hybridized oligonucleotides remaining on the solid support. Such a digestion may yield both more efficient hybridization and cleaner scoring. In this case, it is important that the ends of the oligonucleotides be protected against digestion. At the end of the washing and/or digestion step, the solid support should bear oligonucleotides on which a fragment of sample DNA corresponding to a particular genomic position either is or is not hybridized. Some oligonucleotides will be unhybridized and others will be hybridized.

These oligonucleotides can then be detected as described above, or, instead, the solid support carrying oligonucleotides may be stripped of the hybridizing sample DNA and then rehybridized with labelled oligonucleotides matching each of the original oligonucleotides on the solid support. A second set of washings then ensues, followed by recording or visualization of the labelled, hybridized oligonucleotides.

The recording of hybridization -step follows, and consists of differentiating between the hybridized and unhybridized oligonucleotides in such a way that their hybridization state can be detected. The presence or absence of hybridization for each pair of oligonucleotide sequences is done using any method allowing the recording of the hybridization state.

In one embodiment the hybridization state is recorded (detected) by providing the genomic sample DNA hybridized with the permanently attached oligonucleotides with a label by extending the hybridized DNA by enzymatic action of terminal transferase and providing a label selected from a group consisting of a radioactive, fluorescent, enzymatic, immunochemical, chemical and affinity labels. The chemical label is for example biotin. The labelled extensions are then detected by conventional means corresponding to the label type.

In a specific embodiment of the present invention, the immunochemical label is an antibody capable of detecting the biotin incorporated enzymatically into the DNA hybridized to the oligonucleotide, which is linked to an enzyme catalysing a fluorogenic or chromogenic reaction.

In another embodiment, the hybridization state is detected with a modified mini-sequencing reaction by using oligonucleotides containing standardized tails not corresponding to the genomic position, in which reaction the hybridized fragment serves as the primer to be extended over the tail. The mini-sequencing reaction incorporates labelled nucleotides, which are then detected. In essence, any method that allows distinction between the hybridized and unhybridized states of the oligonucleotides can be used.

In another specific embodiment the oligonucleotides are biotinylated at one end and immobilized on streptavidin-coated polystyrene beads. The detection will be carried out by adding a one base extension to the oligonucleotide sequence, which is not the same base as in the genomic sequence itself. Subsequently, an extension with fluorescently labelled dideoxynucleotides will be used. Because it is known what base is the normal one following the oligonucleotide, some background can be eliminated in this way. The oligonucleotide which will be used for the left flank/right flank (FL/FR) site is actually an inverse oligonucleotide to the other two (i.e. represents the other strand). This is intended to decrease background because the left flank (FL) and right flank (FR) parts of the oligonucleotide are not shared with the left flank (FL) and the right flank (FR) respectively in a left flank/mobile element (FL/ME) and mobile element/right flank (ME/FR).

In another embodiment the oligonucleotides are bound by virtue of a biotin moiety attached during biosynthesis. The key to the method is distinguishing between two states, one in which the probe-sample DNA hybrid is fully double-stranded, and other in which the hybrid is only partially double-stranded because the sample DNA is incompletely homologous to the probe. These two states correspond to situation when a probe for a full insertion site left flank/right flank (FL/ME) for a mobile element is hybridized to sample DNA containing only empty site fragments left flank/right flank (FL/FR); in this case the left flank (FL) segment would hybridize but the mobile element (ME) portion of the probe would not be covered by a region of the probe DNA contiguous with FL. The oligonucleotides correspond to the detection probes and respectively fully complementary or half-length complementary oligonucleotides. A discriminatory hybridization temperature, one that allows the fully-length hybrid to remain annealed while the half-length hybrid would melt, is used in the experiment. To detect the difference between a successfully melt-treated, double-stranded probe/sample hybrid and a single-stranded probe, a dye (PicoGreen® Molecular Probes, Inc) is used. According to the manufacturer PicoGreen® detects specifically dsDNA. The assay mixture likely, after melting, contains a mixture of ssDNA (single-stranded), dsDNA (double-stranded), and half-ss-half-dsDNA. ssDNA-specific nuclease treatment is used to remove the ssDNA.

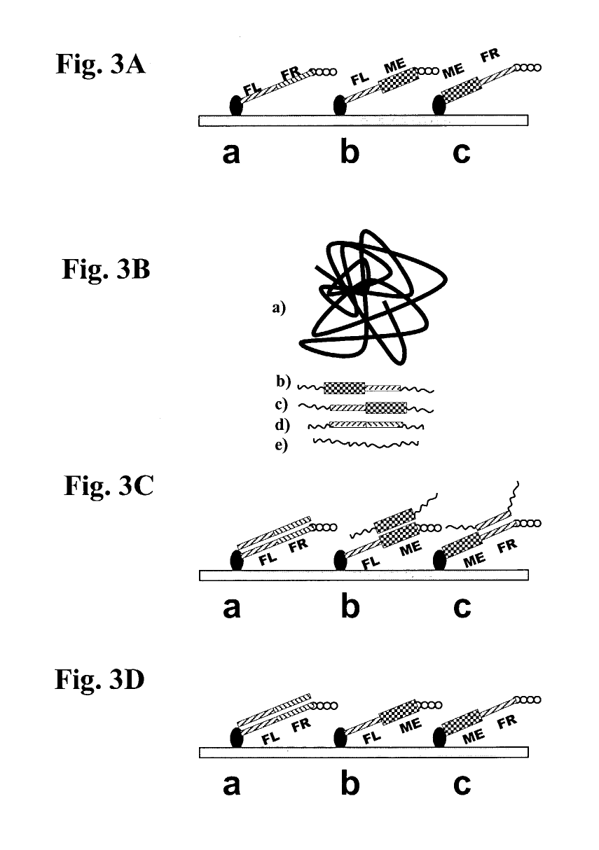

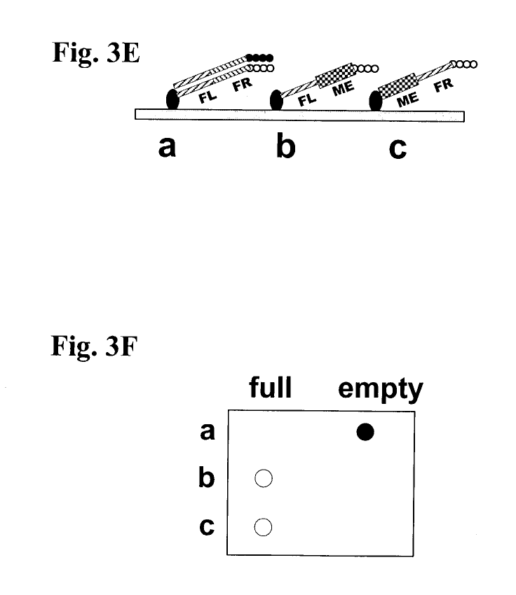

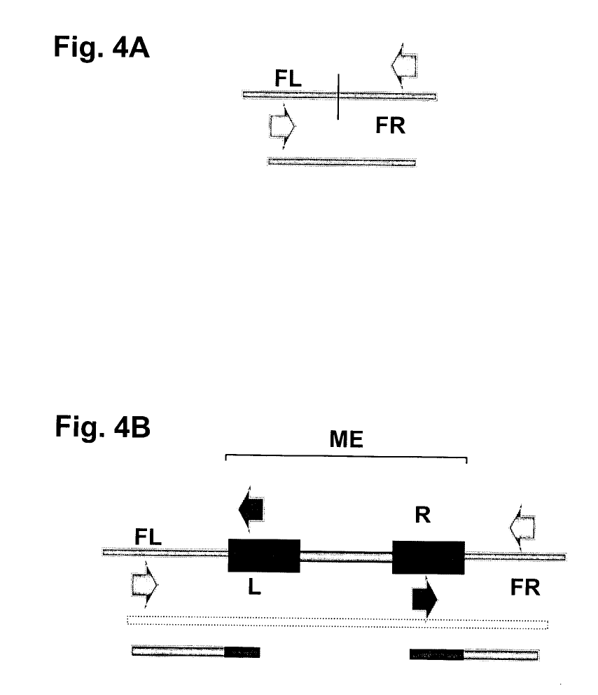

In the detection method schematically shown in FIG. 3 the oligonucleotides are provided with extensions of one or more bases not matching the flanking sequences. The detectable label is incorporated by extension of the hybridized DNA. In FIG. 6 it is schematically shown that a labelled dideoxynucleotide is added which can be incorporated at the end of the oligonucleotide providing the oligonucleotide is hybridized to genomic DNA as template.

The concept of the method and the test kit of the present invention containing scorable oligonucleotides corresponding to particular genomic positions in the genome is quite general. Therefore, any domain in the genome of length feasible for hybridization and screening could be scored. Polymorphisms within these sites could be scored if the hybridization would be followed by digestion with an endonuclease. The endonuclease would cleave mismatches or bubbles within the hybridized sample/oligonucleotide pair. The resulting fragmentation could then destabilize the hybrid and allow release of those cleaved fragments.

The recordable hybridization pattern wherein the presence or absence of hybridization indicates the presence or absence of a mobile element (ME) insertion, respectively, is scored. Co-dominant scoring is carried out per genomic position using the flanking sequences and flanking/mobile element (ME) oligonucleotide(s) as optional paired or parallel sets enabling for a diploid genotype the following alleles to be distinguished: full, empty, failure or null alleles. For co-dominant scoring at least two of the flanking oligonucleotide sequences together with the mobile element (ME) sequence are necessary for the construction of oligonucleotides for identifying the empty and the full site, respectively. Null or failure alleles corresponding to the loss of the genomic position or more precisely to loss of the ability to hybridize to the genomic position, are scored as such when both the empty oligonucleotides and the full oligonucleotides give a no-hybridization response. The data is then analyzed as for conventional co-dominant marker systems.

The scores are recorded as a “difference table” where e.g. accessions are listed vertically and scored genomic positions horizontally across the table. In each cell on the table, a value is placed, 2 for homozygous full/full, 1 for heterozygous and 0 for homozygous empty/empty. Failures or nulls are marked as missing data (−). The data can then be analyzed by methods suited to the specific question. Genetic distances can be estimated from the difference tables using equations of Nei and co-workers (Nei and Li, Proc Natl Acad Sci USA 76:5269-5273, 1979; Saitou and Nei, Mol. Biol. Evol. 4:406-425, 1987). Trees (cladograms) can be constructed by neighbor joining (Saitou and Nei, Mol. Biol. Evol. 4:406-425, 1987), or statistical differences can be estimated with Principal Component Analysis or other standard tests. Software packages exist for this purpose (Bevan and Houlston, Mol. Biotechnol. 17:83-89, 2001; Tores and Barillot, Bioinformatics 17:174-179, 2001).

The hybridization, washing, recording, and scoring of the present invention are all subject to automation. In one embodiment, the processing of the solid support with immobilized oligonucleotides hybridized to sample DNA is carried out in a purpose-built chamber with automated treatment steps. As discussed above the steps including hybridization, post-hybridization treatment, recording of the hybridization state and scoring can be automated.

In a preferred embodiment the test kit includes a DNA chip data collection device (DCD). It is envisaged that the DNA chip DCD will be a portable semi-solid state device into which DNA chips can be loaded, scanned and scored. Development and manufacture of the DNA chip DCD according to methods is well known in the art (U.S. Pat. Nos. 5,445,934, 5,510,270, 5,744,305, 5,700,637).

The hybridized sample DNA is released from the solid support and subsequently hybridized with labelled oligonucleotide sequences corresponding to each of the original oligonucleotide sequences attached to the solid support. It is useful but not essential that the process of development and visualization be reversible, in that the solid support with immobilized oligonucleotides could be returned to its original state and reused.

In the preferred embodiment of the present method for co-dominant scoring the following several steps are comprised.

The oligonucleotides are provided on a solid support comprising more than one set of optionally paired or parallel single stranded oligonucleotide sequences, each of said oligonucleotide sequences comprising one oligonucleotide sequence corresponding to a full site and the other to an empty site, wherein the full site comprises an oligonucleotide sequence being composed of two parts one of which comprises the flanking region of a mobile element (ME) and the other comprises the terminal end of the mobile element (ME) and the oligonucleotide sequence corresponding to the empty site comprises an oligonucleotide sequence composed of two parts one of which is the left flanking region (FL) and the other the right flanking region (FR) surrounding the absent mobile element (ME).

As the first step the sample DNA representing total DNA of the sample is optionally sheared with physical, mechanical or enzymatic means in order to obtain the mobile element (ME) of the organism(s) to be distinguished onto different pieces of DNA.

Thereafter, fragmented sample DNA rendered single stranded is allowed to hybridize with the single stranded oligonucleotide sequences attached to the solid support under conditions which allow the sample DNA to anneal to the oligonucleotide sequences in their full length. Non-hybridized or partly hybridized sample DNA is removed by optional post-hybridization treatments, which may include one or more, washing steps at different stringencies and enzymatic digestion to prevent single stranded nucleotide sequences protruding from the attached probes disturbing labelling and subsequent recording of the results.

The hybridization state is recorded by providing the sample DNA hybridized with the attached oligonucleotide sequences with a recordable label.

Again optional washing steps at different stringencies may be applied before recording the presence or absence of hybridization for each pair of oligonucleotide sequences using any method allowing recording of the hybridization state. The method described above allows scoring of the recordable hybridization pattern wherein the presence or absence of hybridization indicates the presence or absence of a mobile element (ME) in an insertion site. The method is co-dominant.

The invention is described in more detail in the following examples in which the invention is applied to certain plants. These examples should not be interpreted to limit the scope of invention to said exemplified organisms. It is clear to one skilled in the art that the method and test kit can be applied to demonstrate genetic diversity in any organisms and any sample.

### EXAMPLE

### Example 1

Identification of Flanking Sequences for Designing Oligonucleotides

(a) The Sequence Specific Amplified Polymorphism (SSAP) Method (Prior Art)

1. A SSAP reaction is carried out as described (Waugh, et al., Mol. Gen. Genet. 253: 687-694, 1997) in a thermocycler (Applied Biosystems GeneAmp System 9700) using Taq or other thermostable polymerase and reagents as described. The primers consist of one primer designed to correspond to the Long Terminal Repeat (LTR) of BARE-1 with the possible addition of selective bases, as described for SSAP (Waugh, et al., Mol. Gen. Genet. 253: 687-694, 1997) and another primer, which is a PstI SSAP adapter primer. The BARE-1 primer is complementary to the first 19 bases of the element with one extra A selective base at the 3′ end. The PstI adapter primer is GACTGCGTACATGCAG (SEQ ID NO:1). The template DNA is obtained by PstI and MseI digestion of sample DNA such as barley DNA. The DNA is produced by standard means using DNeasy Plant mini kit (Qiagen product 69103).

2. An acryl amide sequencing gel is prepared according to standard procedures (Ausubel et al., Current Protocols in Molecular Biology, John Wiley & Sons, Inc., New York, 1995) to match a standard vertical acryl amide electrophoresis apparatus (Hoeffer SQ3 sequencer, Amersham Pharmacia Biotech catalogue 80-6301-16). An electrophoretic separation is carried out according to the instructions provided for the apparatus. A band that is polymorphic across the accessions, i.e. the band of interest, is chosen. From an accession containing the band of interest, the band is excised from a gel, then macerated in 100 μl TE buffer (Tris-EDTA, 10 mM Tris-HCl, pH 8.0, 1 mM NaEDTA, pH 8.0 as described in Ausubel et al., Current Protocols in Molecular Biology, John Wiley & Sons, Inc., New York, 1995).

3. The DNA in the band of interest, which has been excised from the gel, and eluted in step 2, is PCR-amplified with the original primers as specified in step 1 under the same conditions used for the SSAP in step 1, with 25 cycles using 0.5 μl of the 100 μl eluate from the extracted band as the template.

4. The DNA from the excised and extracted band is sequenced using a commercial sequencing service or alternatively any standard sequencing apparatus (Applied Biosystems ABI Prism 3700 DNA Analyzer) using the manufacturer's reagents and protocols, to determine the sequence of the region flanking the mobile element (ME).

5. From the flank, two nested primers, which are situated as far away from the mobile element (ME) as possible and have a melting temperature matching (for BARE-1 it is 60-65° C.) that of the mobile element (ME), are designed to amplify towards the mobile element (ME) insertion.

6. The primers (prepared in step 5) are used in combination with the PstI digested and adapter ligated DNA from accessions lacking the insertion as seen from the SSAP gel.

7. Using as primers first the outer and then the inner primers from the flank and as a template 0.5 μl of 100 μl from the extracted band a succession of 35 PCR-cycles are carried out.

8. The PCR product is checked for size and yield by electrophoretically separating it on a standard agarose gel, the agarose percentage of which is determined by the expected size. A band of high yield should appear in the last amplification. The product is sequenced as described above and gives the sequences of the original flank and the matching flank from the other side of the integration site of the mobile element (ME). The insertion is legitimated by designing primers to the other flank and demonstrating amplification of the empty site from the accessions lacking the SSAP band.

9. The sequences of the flanks obtained by the steps described above are used to design the oligonucleotides to be attached to the solid support.

(b) The Genome Walking Strategy (Prior Art)

The method is carried out essentially following the method of Siebert et al., Nucl. Acids Res. 23: 1087-1088, 1995) using the GenomeWalker™ kit (BD Biosciences Clonetech, Palo Alto, USA), according to the manufacturer's instructions.

The method follows the steps described in Example 1 a for determination of one flank.

Following this step, genome-walker libraries are created with restriction enzymes, adapters ligated and adapter-primers as specified for the kit in combination with the flanking region primers. The sequence of the major band from each library should coincide to the same site. In other words, the flanking sequence at which the mobile element (ME) is inserted in the alleles containing the mobile element (ME) should be the same. Thereafter, steps 8 and 9 described in Example 1 a are repeated.

The polynucleotides corresponding to the unique flanking regions of the mobile elements (MEs) are identified using the methods described above. The primers corresponding respectively to the Long Terminal Repeat (LTR) and flanking regions are used to carry out Retrotransposon-based Microsatellite Amplified Polymorphism (RBIP) amplifications as described by Flavell, et al. (Plant J. 16: 643-650, 1998). Genotypes corresponding to the range of genotypes likely to be analyzed and distinguished for the particular application are subjected to RBIP analysis. The primer pairs, which effectively distinguish these genotypes are chosen for further development. The flanking regions in each case are PCR amplified using the mobile element (ME) and flank primers, the regions sequenced, and polynucleotides are synthesized on the basis of their sequences.

### Example 2

Detection of Genomic Variation in Maize

The present method is used for detecting polymorphism in maize (Zea mays L.), using mobile element Zeon-1, as present in nucleotide database accession AF090447 (346296 bp) for the 22 kDa alpha zein gene cluster within inbred line BSS53, and on the Heartbreaker (Hbr) Miniature Inverted Repeat Transposable Element (MITE), an element whose polymorphic insert and use as a molecular marker is described in Zhang, et al., Proc. Natl. Acad. Sci. USA 97(3): 1160-1165, 2000 and Casa, et al., Proc. Natl. Acad. Sci. USA 97(18): 10083-10089, 2000.

a)

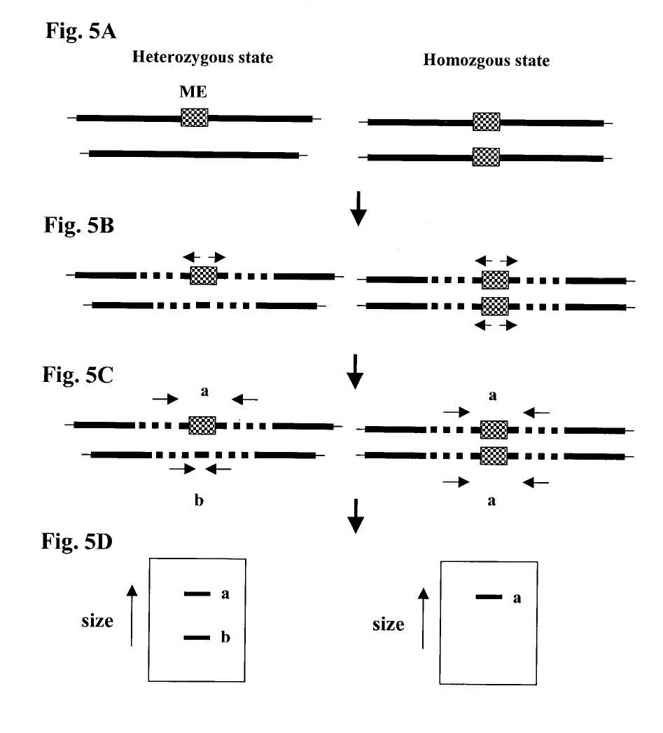

According to Zhang, et al., Proc. Natl. Acad. Sci. USA 97(3): 1160-1165, 2000, one Hbr7 (accession number AF203730) (SEQ ID NO: 2) (prior art) element has the genomic flanking sequences: CGGACGCGCCAGCCAT (SEQ ID NO:3) on the left and CATCCTTTGCTTTGGT (SEQ ID NO:4) on the right (FIG. 5 in Zhang et al., Proc. Natl. Acad. Sci. USA 97(3): 1160-1165, 2000), the CAT being a terminal direct repeat generated by insertion of the element.

Given these sequences, a left flank/mobile element (FL/ME) oligonucleotide can be designed as: 5′ CGCCAGCCATgggtctgttt 3′ (SEQ ID NO: 5) (Hbr in lower case), with an estimated Tm of 58.4° C. for the perfectly-hybridized probe and an estimated Tm for the half-hybrid of 37.5° C. (FL) and 29.0° C. (ME) respectively.

The mobile element/right flank (ME/FR) oligonucleotide can be designed as: 5′ aaacagggccCATCCTTTGC 3′ (SEQ ID NO:6) with an estimated Tm of 58.5° C. for the perfectly hybridized probe and a Tm of 34.4° C. (ME) and 30.3° C. (FR) for the half-hybrids.

The left flank/right flank (FL/FR) oligonucleotide can be designed as: 5′ GCGCCAGCCATCCTTTGC 3′ (SEQ ID NO:7) with an estimated Tm of 58.0° C. for the perfect hybrid in the case of an empty site that has never had a previous Hbr MITE insertion at this point. Because MITE elements are thought to excise in the same way as DNA transposons, a second form of this genomic position may exist in some plant accessions reflecting the excision event leaving behind the double-repeat “footprint” (in bold). This would be: 5′ GCGCCAGCCATCATCCTTTGC 3′ (SEQ ID NO:8) and would have a Tm of 62.6° C.

These oligonucleotides are synthesized, optionally end-protected, and attached to the chip (solid support) and represent a single genomic position for maize. Oligonucleotides for an additional 50 or more genomic positions are derived for the Heartbreaker or other Miniature Inverted Repeat Transposable Element (MITE) using this approach, based on sequencing of flanks and elements as described (Zhang, et al., Proc. Natl. Acad. Sci. USA 97(3): 1160-1165, 2000; Casa et al., Proc. Natl. Acad. Sci. USA 97(18): 10083-10089, 2000).

b)

In another example, from maize database accession AF090447, containing a 346296 bp contiguous region including the Zea mays 22 kDa alpha zein gene cluster, one finds a Zeon-1 LTR retrotransposon. The 100 bp left flanking (FL) region of this element is SEQ ID NO:9 and does not produce any matches to repetitive elements in maize using BLAST. The 100 bp right flanking (FR) region is SEQ ID NO:10 and does not produce any matches to repetitive elements in maize using BLAST.

The left end of the Zeon-1 LTR is 5′ TGTTGGGGGCCTTCGGCTTCCGAAGGTCCT CAAAAACAAGATTTAACTG 3′ (SEQ ID NO:11) and right end of the Zeon-1 LTR is 5′ TGTGTTGCCTTGTTCTTAATTCATAGCATTTGAGAACAAGTCCCCAACA 3′ (SEQ ID NO: 12) with 8 bp terminal inverted repeats within the LTR being underlined.

The left flank/mobile element (FL/ME) joint at this genomic position is CTAACCTGA AAGGTACTGTTGGGGGC . . . (SEQ ID NO:13) and mobile element/right flank (ME/FR) joint is . . . AAGTCCCCA - - - ACAGGTACCCACTGGTAGCCCT (SEQ ID NO:14) where the direct repeats generated by insertion are displayed in bold, the ends of the left and right LTRs underlined, the intervening Zeon-1 sequence represented by dots.

Based on these sequences, a left flank/mobile element (FL/ME) oligonucleotide can be designed as 5′ TGAAAGGTACTGTTGGGGGC 3′ (SEQ ID NO:15) with Tm of 54.4° C. for the fully hybridized oligonucleotide, and respectively 25.4° C. and 36.9° C. for the left and right half-hybrids.

The mobile element/right flank (ME/FR) oligonucleotide can be designed as 5′ GTCCCCAACAGGTACCCACTG 3′ (SEQ ID NO:16) with Tm values of 54.7° C. for the full oligonucleotide, 31.5° C. for the ME half-hybrid, and 31.2° C. for the FR half-hybrid.

The left flank/right flank (FL/FR) oligonucleotide can be designed as 5′ CTGAAAGGTACCCACTGGTAGC 3′ (SEQ ID NO: 17) with a Tm of 53.7° C. It should be noted that, as for other retrotransposon left flank/right flank (FL/FR) oligonucleotides, the direct repeat generated upon insertion is present in only one, and not two copies in the un-interrupted native site.

These oligonucleotides are synthesized, optionally end-protected, and attached to the chip and represent a single genomic position for maize (FIG. 3). Oligonucleotides for an additional 50 or more genomic positions for other LTR retrotransposons can be derived as given in Example 1.

Furthermore, oligonucleotides left flank/mobile element (FL/ME) (SEQ ID NO:26), right flank/left flank (FR/FL) (SEQ ID NO:27) and right flank/mobile element (FR/ME) (SEQ ID NO:22s) are used in the method presented in FIG. 6. These oligonucleotides have been chosen so that different nucleotides would be incorporated as the next nucleotide in the dideoxy extension (respectively A, G, C and T or U).

### Example 3

Preparation of the Sample DNA

DNA is prepared by the CTAB method (Ausubel, et al., Current Protocols in Molecular Biology, John Wiley & Sons, Inc., New York, 1995) and RNase-treated as described therein. Alternatively, commercial preparation systems (Qiagen's kits, DNeasy, or the Genomic tips for clinical samples) are used. The DNA is sonicated without any prepreparation step by use of a sonicator. Sonication is most efficient at a high DNA concentration, such as 10 μg/μl. The DNA is sonicated with an appropriate apparatus (B. Braun Biotech International Labsonic) having an output frequency of 20 kHz and a power maximum of 350 watts and a needle probe probel 40 TL (catalogue number 853 811/5). The sonication is carried out with a 50% duty cycle and approximately 10-20% power level (“low”), preferably on ice, for 10 to 20 minutes, or for such time as there is a clear reduction in sample viscosity and the DNA fragment size is reduced to ca. 500 bp or less. The sample DNA is sheared to small (ca. 500 bp or less) pieces by any means, including digestion with a frequent (such as 4-base) restriction enzyme or (preferred) sonication. The purpose of shearing is to physically separate the particular genomic positions to be scored onto different pieces of DNA to increase the efficiency of the process.

### Example 4

Recording Hybridization

The hybridization recording step follows, and consists of differentiating between hybridized and unhybridized oligonucleotides in such a way that their hybridization state can be detected. In one embodiment, (FIG. 3E) the hybridized genomic DNA is extended by enzymatic action of a terminal transferase, using either radio-labelled, fluorescent, or chemically labelled (e.g., biotin) oligonucleotides. The labelled extensions are then detected by conventional means corresponding to the label type. In another embodiment, the oligonucleotides contain standardized tails at their 5′ ends not corresponding to genomic position. The hybridized fragment then serves as the primer to be extended in the typical 5′-->3′ direction over the tail in a modified mini-sequencing reaction. The mini-sequencing reaction incorporates labelled nucleotides, which are then detected. In essence, any method, which allows distinction between the hybridized and unhybridized states of the oligonucleotides, can be used. It is useful but not essential that the process of development and visualization be reversible, in that the chip could be returned to its original state and reused.

### Example 5

Scoring of the Recorded Hybridization Pattern

The recorded hybridization pattern is then scored. The scoring is done per genomic position, using the flanking oligonucleotides and the flank/mobile element (ME) oligonucleotides as sets. This enables the following alleles to be distinguished: full, empty, and null or failure. The data are then analyzed as for conventional co-dominant marker systems. The scores are recorded as a “difference table” where e.g. accessions are listed vertically and scored genomic positions horizontally across the table. In each cell on the table, a value is placed, 2 for full/full, 1 for heterozygous and 0 for empty/empty. Failures or nulls are marked as missing data (−). The data can then be analyzed by methods suited to the specific question. Genetic distances can be estimated the difference tables using equations of Nei and co-workers (Nei and Li, Proc. Natl. Acad. Sci. USA 76:5269-5273, 1979; Saitou and Nei, Mol. Biol. Evol. 4:406-425, 1987). Trees (cladograms) can constructed by neighbour-joining (Saitou and Nei, Mol. Biol. Evol. 4:406-425, 1987), or statistical differences can be estimated with Principal Component Analysis or other standard tests. Software packages exist for this purpose. Pedigree analysis can be performed on the data as well. Methods and software for this is known in the field, e.g. Kindred and Gap (Bevan and Houlston, Mol. Biotechnol. Jan; 17(1):83-9, 2001; Tores and Barillot, Bioinformatics 2001 February;17(2):174-9, 2001).

The hybridization, washing, recording and scoring according to the present disclosure are all subject to automation. In one embodiment, the processing is carried out in a purpose-built chamber with automated treatment steps.

### Example 6

Detection of Genomic Variation in Barley

The present method is used for detecting polymorphism in barley (Hordeum vulgare L.), using mobile element BARE-1. The polymorphisms were detected using Inter-Retrotransposon Amplified Polymorphism (IRAP) and Retrotransposon Microsatellite Amplified Polymorphism (REMAP), in screening cultivars of spring and winter barley with the IRAP and REMAP methods and BARE-1 LTR primers.

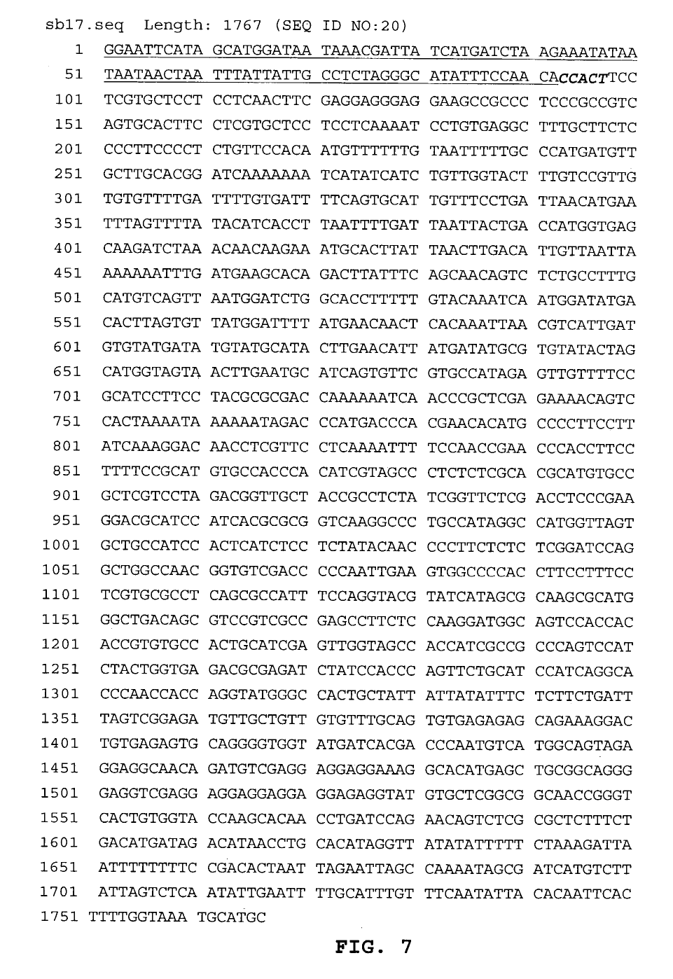

Using primer 7286 (GGAATTCATAGCATGGATAATAAACGATTATC) (SEQ ID NO:18) and (CTC)₉C (SEQ ID NO:19) in REMAP, a polymorphic band was identified that was present only in spring barley accessions but not in winter barley accessions. The band was excised from the ethidium bromide stained agarose gel, cloned, and sequenced. The 1767 nt sequence sb17 (SEQ ID NO: 20) is presented in FIG. 7. The LTR of the BARE-1 insertion is underlined. It represents the end of an LTR inverted with respect to the sense direction of the open reading frame. The predicted 5 bp direct repeat generated by the insertion, CCACT, is in bold italics in FIG. 7.

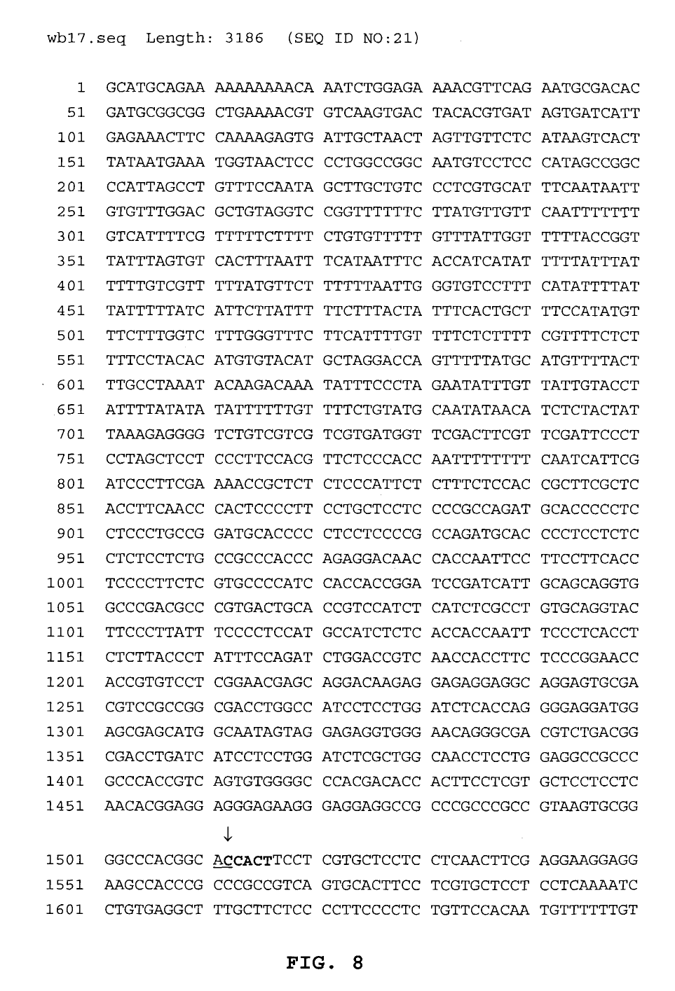

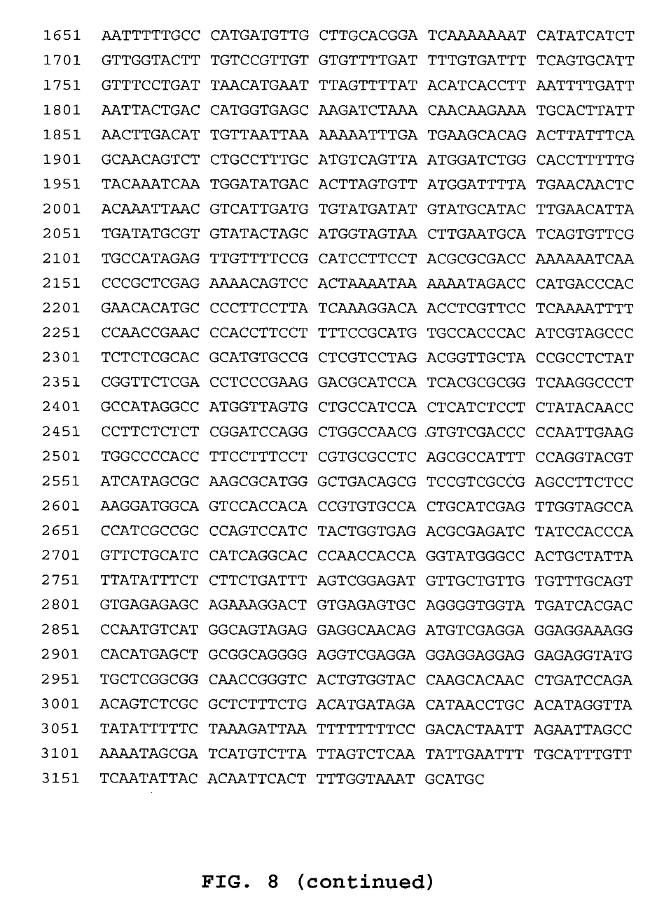

The region corresponding to this band was likewise cloned from a winter barley accession. Winter barley lacks the BARE-1 insertion at this genomic position. The 3186 nt sequence wb17 (SEQ ID NO: 21) is presented in FIG. 8. This sequence is almost completely identical with the sb17 sequence (SEQ ID NO:20) above, except that it lacks the BARE-1 sequence. The insertion site of the BARE-1 in sb17 (SEQ ID NO:20) is marked with an arrow in the wb17 (SEQ ID NO:21) sequence in FIG. 8, and is located between nucleotides 1511 and 1512 in wb17 (SEQ ID NO:21). These flanking nucleotides are underlined. The 5 bp sequence which forms the direct repeat in sb17 (SEQ ID NO: 20) is in bold in FIG. 8.

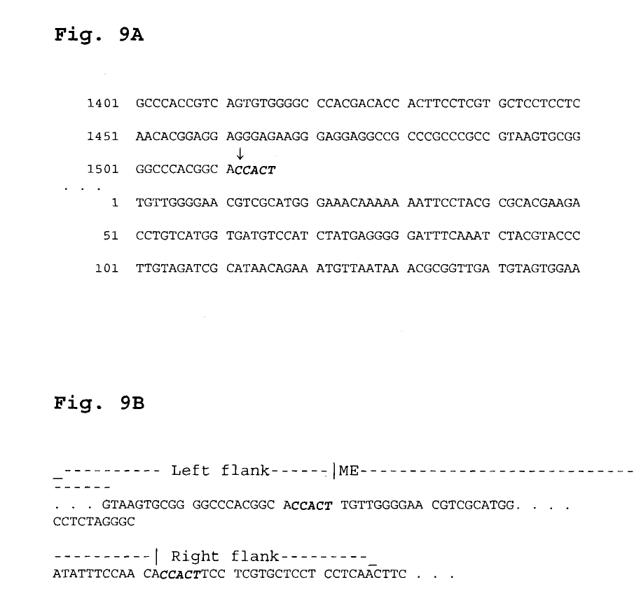

Given the sb17 sequence (SEQ ID NO: 20), a 23 nt right flank/mobile element (FR/ME) oligonucleotide can be designed as 5′ tatttccaacaCCCACTTCCTCG 3′ (SEQ ID NO: 22) (BARE-1 in lower case), with an estimated Tm of 57.8° C. for the perfectly-hybridized probe and an estimated Tm for the half-hybrids of 38.2° C. (FR) and 28.6° C. (ME) respectively. The mobile element/left flank (ME/FL) flanking region can be predicted from the BARE-1 LTR, the predicted 5 bp direct repeat, and wb17 (SEQ ID NO:21) sequences respectively in the following manner (FIG. 9a). The SEQ ID NO: 23 represents the first 100 nt of the inverse-orientation BARE-1 LTR and c.a. 100 nt predicted for the sb17 (SEQ ID NO: 20) 5′ joint region. The flanks and the inserted mobile element (ME) in summary can be displayed as in FIG. 9b.

Left flank/mobile element (FL/ME) is GTAAGTGCGGGGCCCACGGCA CCACTTGTTGGGGAACGTCGCATGG (SEQ ID NO:24) and right flank/mobile element (FR/ME) CCTCTAGGGCATATTTCCAACACCACTTCCTCGTGCTCCTCCTCAACTTC (SEQ ID NO:25).

These oligonucleotides are synthesized, optionally end-protected, and attached to the chip and represent a single genomic position for maize. Oligonucleotides for an additional 50 or more genomic positions are derived and treated in a similar manner.

The oligonucleotide e.g. mobile element/right flank (ME/FR) is biotinylated at one end. This allows the attachment to the solid support. The sheared DNA is introduced and allowed to hybridize at a “non-permissive” temperature, i.e. one at which the half-hybrid does not stick. One of each of the four ddNTPs, fluorescein labeled, is added to a tube. The ddNTP, which corresponds to the next base following the end of the oligonucleotide is incorporated into the end of the oligonucleotide. The other three reactions are controls that are predicted not to give an incorporated ddNTP. This controls for specificity of the recognition. The reaction is set up in a cycler, and goes through approximately 5 rounds of melting, annealing, and extension to increase sensitivity. Then the biotin label is captured on the streptavidine styrene beads, and the fluorescence from fluorescein measured.

In this embodiment the oligonucleotide is extended, rendering it not reusable, rather than the added genomic DNA. This takes away the need for a nuclease trimming step as the oligonucleotide is hybridized, and may make the shearing unnecessary.

In a more complex setting, the reaction is carried out e.g. on a microtiter plate with the oligonucleotides pre-attached to the plate, one per well.

### Example 7

Discrimination of the Hybridization States

The oligonucleotides are bound by virtue of a biotin moiety attached during biosynthesis. The key to the method is distinguishing between two states, one in which the probe-sample DNA hybrid is fully double-stranded, and the other in which the hybrid is only partially double-stranded because the sample DNA is incompletely homologous to the probe. These two states correspond to the situation when a probe for a full insertion site left flank/mobile element (FL/ME) for a mobile element (ME) is hybridized to sample DNA containing only empty site fragments left flank/right flank (FL/FR); in this case the left flank (FL) segment would hybridize but the mobile element (ME) portion of the probe would not be covered by a region of the probe DNA contiguous with left flank (FL).

The oligonucleotides corresponded, as detailed below, to the detection probes and respectively fully complementary or half-length complementary oligonucleotides. A discriminatory hybridization temperature, one that allows the full-length hybrid to remain annealed while the half-length hybrid would melt, was used in the experiment.

(a) Sample DNA

As sample DNA, oligonucleotides corresponding, as detailed below, to the detection probes and respectively fully complementary or half-length complementary oligonucleotides were used. Three different single stranded sequences represented three different genomic states of the sample DNA, i.e. mobile element (ME) (F0740; SEQ ID NO: 30), Right Flank/Mobile Element (FR/ME) (F0739; SEQ ID NO: 29) and Right Flank/Left Flank (FR/FL) (F0738; SEQ ID NO: 28) i.e. an empty site.

Double-stranded λDNA was used as standard.

DNA Samples

SEQ ID NO: 28

| | | |
|---|---|---|
| F0738 | 5′ CAC GGC ACC ACT TCC TCG TGC 3′ | FR/FL |

SEQ ID NO: 29

| | | |
|---|---|---|
| F0739 | 5′ GAG GAA GTG GGT GTT GGA AAT A 3′ | FR/ME |

SEQ ID NO: 30

| | | |
|---|---|---|
| F0740 | 5′ CTC CTT CAC CCT GTT GGA AAT A 3′ | ME |

(b) Probes

Two single stranded oligonucleotides represented Right Flank/Left Flank (FR/FL) (E2458; SEQ ID NO: 27) and Right Flank/Mobile Element (FR/ME) (E2460; SEQ ID NO: 22):

SEQ ID NO: 27

| | | |
|---|---|---|
| E2458 | 5′ AGC ACG AGG AAG TGG TGC CGT G 3′ | FR/FL |

SEQ ID NO: 22

| | | |
|---|---|---|
| E2460 | 5′ TAT TTC CAA CAC CCA CTT CCT CG 3′ | FR/ME |

The oligonucleotides that were used as probes were biotinylated.

(c) Method

Hybridization was done using the following reaction mix. Different concentrations (1 μg, 0.5 μg, 0.1 μg, 50 pg and 25 pg) of the biotinylated oligonucleotide (E2458 or E2460) and the other oligonucleotide (F0738, F0739 or F0740) were used. The amount of MQ-water was adjusted so that the total reaction volume was equal to 50 μl.

Reaction mix:

$$\begin{matrix}x μ g & biotinylated oligonucleotide ( E2458 or E2460 ) \\ x μ g & other oligonucleotide ( F0738 , F0739 or F0740 ) \\ 5 μ1 & 10 \times TE + 2000 mM NaC1 \\ x μ1 & MQ - water \\ 50 μ1 & total volume \end{matrix}$$

Attachment of the Biotinylated Oligonucleotides to the Solid Support

Different concentrations, 1 μg, 0.5 μg, 0.1 μg, 50 pg and 25 pg, of the biotinylated oligonucleotide (E2458 or E2460) were attached to the solid support. A streptavidin-coated plate (DELFIA Streptavidin microtitration plate, Wallac Oy) was used as a solid support. Oligonucleotides and MQ-water according to the reaction mix above were pipetted to streptavidin coated plate and mixed for 30 minutes on a plate shaker.

Hybridization

The other oligonucleotide (F0738, F0739 or F0740) at the same concentration as the biotinylated oligonucleotide and 1×TE+200 mM NaCl were added. The reaction mix was heated to 65° C. in a water bath and incubated for 30 minutes. After incubation the reaction mix was allowed to cool to room temperature (25° C.) for approximately 15 min.

Hybridized oligonucleotide pairs were:

| | |
|---|---|
| 1A | FR/ME, ME complement alone (F0740, E2460) |
| 1B | FR/ME, FR/ME complement (F0739, E2460) |
| 1C | FR/FL, FR/FL complement (F0738, E2458) |

Exonuclease T Treatment

After hybridization the hybridized oligonucleotide pairs were treated with Exonuclease T to remove free ssDNA. The treatment was carried out on the plate with the attached oligonucleotides.

Reaction mix:

$$\begin{matrix}50 μ 1 & hybridized oligonucleotide pairs ( 1 A , 1 B or 1 C ) \\ 10 μ 1 & 10 \times NE buffer \\ 0 \text{,} 2 μ1 & Exonuclease T ( 5 U / μ1 ) \\ 39 \text{,} 8 μ1 & MQ - water \\ 100 μ1 & total volume \end{matrix}$$

The reactions were incubated 1 hour in 25° C. (room temperature). The reactions were heated to 45° C. and incubated 2 minutes. This temperature is non-permissive for half-hybrid(s).

Detection

To detect the difference between a successfully melt-treated, double-stranded probe/sample hybrid and a single-stranded probe, the PicoGreen® dye (Molecular Probes, Inc) was used. According to the manufacturer PicoGreen® detects specifically dsDNA. The assay mixture was likely, after melting, to contain a mixture of ssDNA (single-stranded), dsDNA (double-stranded), and half-ss-half-dsDNA. It was not possible to get specific information from the manufacturer on exactly how much fluorescence could be expected from the ssDNA. In the present experiment clean results could be obtained only by a combination of melting and ssDNA-specific nuclease treatment to remove the ssDNA.

Fresh PicoGreen® working solution was prepared by making a 200-fold dilution in 1×TE from PicoGreen® stock. 100 μl of picogreen working solution was added to each well and then mixed in plate shaker. Picogreen working solution of 100 μl and 100 μl 1×TE were used as a blank. Samples were incubated for 5 minutes in the dark. After incubation, the fluorescence of samples was measured (excitation 485 nm, emission 535 nm). The gain setting of the plate reader was set to a value that optimized the signal-to-background level.

Results

The results of the hybridization are presented in Table 1.

TABLE 1
(Average-zero) and standard deviation Zero wells averaged 244

| | 1A | | 1B | | 1C | |
|---|---|---|---|---|---|---|
| | Average | Stdev | Average | Stdev | Average | Stdev |
| 1 μg + 1 μg | 29148 | 1244 | 42922 | 2559 | 41675 | 942 |
| 0.5 μg + 0.5 μg | 28548 | 1346 | 38064 | 305 | 36094 | 3687 |
| 0.1 μg + 0.1 μg | 9068 | 394 | 26329 | 3333 | 23642 | 1967 |
| 50 pg + 50 pg | 3940 | 44 | 9914 | 98 | 12507 | 888 |
| 25 pg + 25 pg | 1923 | 133 | 3950 | 175 | 5834 | 89 |

Conclusions

The half-hybrid (1A) can be discriminated from the full hybrids (1B, 1C) by a two- to three-fold difference in fluorescence response. The half- and full-hybrid distinction is consistent and sufficient for application.

### Example 8

Discrimination of the Hybridization States in a Genomic DNA Background

The experiment is carried out as described in Example 8, except that an oligonucleotide mixed with sheared barley DNA is used as sample DNA.

(a) Sample DNA

As sample DNA, we used barley DNA (cultivar Bomi) sheared by sonication and oligonucleotides corresponding, as detailed below, to the detection probes and respectively fully complementary or half-length complementary oligonucleotides. Three different single stranded sequences representing three different genomic states of the sample DNA, i.e. mobile element (ME) (F0740; SEQ ID NO: 30), Right Flank/Mobile Element (FR/ME), (F0739; SEQ ID NO: 29) and Right Flank/Left Flank (FR/FL) (F0738; SEQ ID NO: 28), i.e. an empty site.

Double-stranded □DNA was used as standard.

DNA-Samples

SEQ ID NO:28

| | | |
|---|---|---|
| F0738 | 5′ CAC GGC ACC ACT TCC TCG TGC 3′ | FR/FL |

SEQ ID NO:29

| | | |
|---|---|---|
| F0739 | 5′ GAG GAA GTG GGT GTT GGA AAT A 3′ | FR/ME |

SEQ ID NO:30

| | | |
|---|---|---|
| F0740 | 5′ CTC CTT CAC CCT GTT GGA AAT A 3′ | ME |

Sheared Barley DNA was Added in Each Reaction

(b) Probes

Two single stranded oligonucleotides represented Right Flank/Mobile Element (FR/ME) (E2460; SEQ ID NO:22) and Right Flank/Left Flank (FR/FL) (E2458; SEQ ID NO:27).

SEQ ID NO:27

| | | |
|---|---|---|
| E2458 | 5′ AGC ACG AGG AAG TGG TGC CGT G 3′ | FR/FL |

SEQ ID NO:22

| | | |
|---|---|---|
| E2460 | 5′ TAT TTC CAA CAC CCA CTT CCT CG 3′ | FR/ME |

The oligonucleotides that were used as probes biotinylated.

(c) Method

Hybridization was done using the following reaction mix. Different concentrations (1 μg, 0.5 μg, 0.1 μg, 50 pg and 25 pg) of the biotinylated oligonucleotide (E2458 or E2460) and the other oligonucleotide (F0738, F0739 or F0740) were used. The amount of MQ-water was adjusted so that the total reaction volume was equal to 50 μl.

$$\begin{matrix}x u g & biotinylated oligonucleotide \\ x u g & other oligonucleotide \\ 10 n g & sheared barley DNA \\ 5 u 1 & 10 \times TE + 2000 m M NaC1 \\ x u 1 & MQ - water \\ 50 u 1 & total volume \end{matrix}$$

Attachment of the Biotinylated Oligonucleotides to the Solid Support

Different concentrations, 50 pg and 100 pg, of the biotinylated oligonucleotide (E2458 or E2460) were attached to the solid support. A streptavidin coated plate (DELFIA Streptavidin microtitration plate, Wallac Oy) was used as a solid support. Oligonucleotides and MQ-water according to the reaction mix above were pipetted to streptavidin coated plate and mixed for 30 minutes on a plate shaker.

Hybridization

Before use sheared barley DNA was heated to 96° C. for 5minutes and chilled immediately on ice. Barley DNA and the other oligonucleotide (F0738, F0739 or F0740) at the same concentration as a biotinylated oligonucleotide were mixed and 1×TE+200 mM NaCl was added. The reaction mix was heated to 65° C. in a water bath and incubated for 30 minutes. After incubation the reaction mix was allowed to cool to room temperature (25° C.) for approximately 15 min.

Hybridized oligonucleotide pairs were:

| | |
|---|---|
| 1A | FR/ME, ME complement alone (F0740, E2460) |
| 1B | FR/ME, FR/ME complement (F0739, E2460) |
| 1C | FR/FL, FR/FL complement (F0738, E2458) |

Exonuclease T Treatment

After hybridization the hybridized oligonucleotide pairs were treated with Exonuclease T to remove free ssDNA. The treatment was carried out on the plate with the attached oligonucleotides.

Reaction mix:

$$\begin{matrix}50 μl & hybridized product ( 1 A , 1 B or 1 C ) \\ 10 μl & 10 \times NEbuffer \\ 0 , 2 μ1 & Exonuclease T ( 5 U / μl ) \\ 39 , 8 μ1 & MQ - water \\ 100 μ1 & total volume \end{matrix}$$

The reactions were incubated 1 hour in 25° C. (room temperature). The reactions were then heated to 45° C. and incubated for 2 minutes. This temperature is non-permissive for a half-hybrid(s).

Detection

Fresh PicoGreen® (Molecular Probes, Inc.) working solution was prepared by making 200-fold dilution in 1×TE from PicoGreen® stock.100 μl of PicoGreen® working solution was added to each well and mixed in plate shaker. PicoGreen® working solution of 100 μl and 100 μl 1×TE were used as a blank controls. Samples were incubated for 5 minutes in dark. After incubation, the fluorescence of each sample was measured (excitation 485 nm, emission 535 nm). The gain setting of the plate reader was set to a value that optimized the signal-to-background level.

Results

The results of the hybridization are presented in Table 2.

TABLE 2

| | 1A | | 1B | | 1C | |
|---|---|---|---|---|---|---|
| | Average | Stdev | Average | Stdev | Average | Stdev |
| 50 pg + 50 pg | 6778 | 145 | 17934 | 996 | 22944 | 618 |
| 100 pg + 100 pg | 13823 | 624 | 336504 | 323 | 40539 | 365 |
| 50 pg + 50 pg + DNA | 6473 | 219 | 15872 | 295 | 19281 | 289 |
| 100 pg + 100 | 14173 | 964 | 36346 | 268 | 39674 | 730 |
| pg + DNA | | | | | | |

Conclusions

The half-hybrid (1A) can be distinguished from the full hybrids (1B and 1C) by a two- to three-fold difference in fluorescence response. The presence of a 100 or 200-fold excess genomic DNA (10 ng) did not affect the signal; the results with and without the genomic DNA (Example 7) are not statistically distinct. This indicates that the presence of partially hybridizing genomic sequences do not interfere with the correct, fully-hybridizing oligonucleotide in solution finding its immobilized target on the solid support. This is particularly critical in the case of the right flank/mobile element (FR/ME) pair, 1B, where the abundance of BARE-1 LTRs in the genome would be expected to compete for binding with the full-length right flank/mobile element (FR/ME).

It will be clear to those having skill in the art that many changes may be made in the above-described details of preferred embodiments of the present invention without departing from the underlying principles thereof. The scope of the present invention should therefore be determined only by the following claims.

What is claimed is:

1. A method for demonstrating genetic identity, genetic diversity, genomic variations, polymorphisms, allelic variation or co-dominant scoring within a defined population pool, comprising the steps of:
(a) allowing unlabeled, single-stranded, sample DNA representing total DNA of a sample to hybridize with one or more different sets of oligonucleotide sequences, each oligonucleotide sequence representing a full or an empty integration site of at least one mobile element (ME) and each oligonucleotide sequence further being attached to a defined identifiable location on a solid support;
(b) providing post-hybridization treatments in order to remove sample DNA not fully hybridized to the solid support attached oligonucleotide sequences; and
(c) providing a hybridization product with a recordable label, said label allowing hybridization pattern to be recorded and thereby the presence or absence of a full or an empty integration site of at least one mobile element (ME) to be scored.

2. The method according to claim 1, wherein for co-dominant scoring at least one set of paired or parallel oligonucleotide sequences is provided for each homologue to be scored in the population pool.

3. The method according to claim 1, wherein the hybridization pattern recorded for oligonucleotide sequences representing a full or an empty integration site is used for co-dominant scoring.

4. The method according to claim 1, wherein hybridization takes place under conditions which allow the unlabeled, single-stranded sample DNA to anneal to the oligonucleotide sequences being attached to the solid support.

5. The method according to claim 1, wherein the post-hybridization treatments are carried out under conditions releasing all sample DNA which has not fully hybridized with the oligonucleotide sequences being attached to the solid support.

6. The method according to claim 1, wherein the hybridization pattern is recorded by providing the sample DNA after the hybridization and post-hybridization treatments with a recordable label.

7. The method according to claim 1, wherein the sample DNA after the hybridization and post-hybridization treatments is released from the solid support and subsequently hybridized with labelled oligonucleotide sequences fully corresponding to each of the oligonucleotide sequences which were attached to the solid support.

8. The method according to claim 1, wherein the recording is reversible and the solid support is returned to its original state for reuse.

9. The method according to claim 1, wherein the steps including hybridization, post-hybridization treatment, recording and scoring are automated.

10. The method according to claim 1, wherein the sample DNA is fragmented.

11. The method according to claim 10 wherein hybridization takes place under conditions which allow the unlabelled, single-stranded, fragmented sample DNA to anneal to the oligonucleotide sequences being attached to the solid support.

12. The method according to claim 1, wherein the one or more different sets of oligonucleotide sequences are paired.

13. The method according to claim 1 wherein the one or more different sets of oligonucleotide sequences are parallel.

14. The method according to claim 1, comprising the steps of:
(a) providing a solid support comprising more than one sets of oligonucleotide sequences, each set further comprising at least one oligonucleotide sequence representing a full integration site and one oligonucleotide sequence representing an empty integration site;
(b) shearing the sample DNA representing the total DNA with physical, mechanical or enzymatic means in order to obtain the mobile elements (MEs) onto different pieces of DNA;
(c) rendering said sheared sample DNA single-stranded and allowing said single-stranded sample DNA fragments to hybridize with the single-stranded oligonucleotide sequences attached to the solid support;
(d) providing post-hybridization treatments including removal of single-stranded sample DNA which is not fully hybridized to the oligonucleotide sequences attached to the solid support using washing treatment at different stringencies to remove single-stranded sample DNA fragments not fully corresponding to the attached polynucleotide sequences;
(e) recording a hybridization pattern for each set of oligonucleotide sequences using any method capable of demonstrating the hybridization;
(f) scoring the recordable hybridization pattern wherein the presence of hybridization with a solid support attached oligonucleotide sequence representing a full integration site indicates a presence of at least one mobile element (ME), the presence of hybridization with a solid support attached oligonucleotide sequence representing an empty integration site indicates an absence of a mobile element (ME) in a corresponding integration site, and the absence of hybridization indicates that the integration site is lacking.

15. The method according to claim 14, wherein the post-hybridization treatments include digestion treatments.

16. The method according to claim 1 comprising the steps of:
(a) providing a solid support comprising more than one sets of oligonucleotide sequences, each set further comprising at least one oligonucleotide sequence representing a full integration site and one oligonucleotide sequence representing an empty integration site;
(b) rendering said sheared sample DNA single-stranded and allowing said single-stranded sample DNA fragments to hybridize with the single-stranded oligonucleotide sequences attached to the solid support;
(c) providing post-hybridization treatments including removal of single-stranded sample DNA which is not fully hybridized to the oligonucleotide sequences attached to the solid support using washing treatment at different stringencies to remove single-stranded sample DNA fragments not fully corresponding to the attached polynucleotide sequences;
(d) recording a hybridization pattern for each set of oligonucleotide sequences using any method capable of demonstrating the hybridization;
(e) scoring the recordable hybridization pattern wherein the presence of hybridization with a solid support attached oligonucleotide sequence representing a full integration site indicates a presence of at least one mobile element (ME), the presence of hybridization with a solid support attached oligonucleotide sequence representing an empty integration site indicates an absence of a mobile element (ME) in a corresponding integration site, and the absence of hybridization indicates that the integration site is lacking.

17. The method according to claim 16, wherein the post-hybridization treatments include digestion treatments.

18. The method according to the claim 1 comprising the steps of:
(a) providing a solid support comprising more than one sets of oligonucleotide sequences, each set further comprising at least one oligonucleotide sequence representing a full integration site and one oligonucleotide sequence representing an empty integration site;
(b) shearing the sample DNA representing the total DNA with physical, mechanical or enzymatic means in order to obtain the mobile elements (MEs) onto different pieces of DNA;
(c) rendering said sheared sample DNA single-stranded and allowing said single-stranded sample DNA fragments to hybridize with the single-stranded oligonucleotide sequences attached to the solid support;
(d) recording a hybridization pattern for each set of oligonucleotide sequences using any method capable of demonstrating the hybridization;
(e) scoring the recordable hybridization pattern wherein the presence of hybridization with a solid support attached oligonucleotide sequence representing a full integration site indicates a presence of at least one mobile element (ME), the presence of hybridization with a solid support attached oligonucleotide sequence representing an empty integration site indicates an absence of a mobile element (ME) in a corresponding integration site, and the absence of hybridization indicates that the integration site is lacking.

19. The method according to claim 1, comprising the steps of:
(a) providing a solid support comprising more than one sets of oligonucleotide sequences, each set further comprising at least one oligonucleotide sequence representing a full integration site and one oligonucleotide sequence representing an empty integration site;
(b) rendering said sheared sample DNA single-stranded and allowing said single-stranded sample DNA fragments to hybridize with the single-stranded oligonucleotide sequences attached to the solid support;
(c) recording a hybridization pattern for each set of oligonucleotide sequences using any method capable of demonstrating the hybridization;
(d) scoring the recordable hybridization pattern wherein the presence of hybridization with a solid support attached oligonucleotide sequence representing a full integration site indicates a presence of at least one mobile element (ME), the presence of hybridization with a solid support attached oligonucleotide sequence representing an empty integration site indicates an absence of a mobile element (ME) in a corresponding integration site, and the absence of hybridization indicates that the integration site is lacking.

20. The method according to claim 1, wherein each oligonucleotide sequence attached to the solid support represents a junction in at least one full or one corresponding empty integration site of a mobile element (ME); an oligonucleotide sequence representing a junction in a full integration site further comprising a first and a second distinct sequence region, the first distinct sequence region being a region flanking the mobile element (ME) and the second distinct sequence region being a terminal end of the mobile element (ME); and an oligonucleotide sequence representing a junction in an empty integration site further comprising two distinct sequence regions each composed of flanking regions surrounding the integration site of the mobile element (ME).

21. The method according to claim 20, wherein the first distinct sequence region is longer than the second distinct sequence region.

22. The method according to claim 20, wherein the first and the second distinct sequence regions of the oligonucleotide sequence representing a junction in a full integration site are of equal length.

23. The method according to claim 20, wherein the first and the second distinct sequence regions of the oligonucleotide sequence representing a junction in a full integration site are of varying length.

24. A method for demonstrating genetic identity, genetic diversity, genomic variations, polymorphisms, allelic variation or co-dominant scoring within a defined population pool, comprising the steps of:
a) allowing unlabeled, single-stranded, sample DNA representing total DNA of a sample to hybridize with one or more different sets of oligonucleotide sequences, each oligonucleotide sequence representing a full or an empty integration site of at least one mobile element (ME) and each oligonucleotide sequence further being attached to a defined identifiable location on a solid support;
b) providing a hybridization product with a recordable label, said label allowing hybridization pattern to be recorded and thereby the presence or absence of a full or an empty integration site of at least one mobile element (ME) to be scored.

25. A test kit for demonstrating genetic identity, genetic diversity, genomic variations, polymorphisms, allelic variation or co-dominant scoring in a population pool, said test kit further comprising more than one set of single-stranded oligonucleotide sequences, each oligonucleotide sequence representing a junction in at least one full or one corresponding empty integration site, the oligonucleotide sequence representing junction in a full integration site further comprising a first and a second distinct sequence region, the first distinct sequence region being a flanking region of a mobile element (ME) and the second distinct sequence region being a terminal end of said mobile element (ME) and a oligonucleotide sequence representing the corresponding empty integration site further comprising two flanking regions surrounding said mobile element (ME).

26. The test kit according to claim 25, wherein the first distinct sequence is longer than the second distinct sequence region.

27. The test kit according to claim 25, wherein at least one set of oligonucleotide sequences is provided for each homologue to be scored present in the population pool.

28. The test kit according to claim 25, wherein the oligonucleotide sequences are paired.

29. The test kit according to claim 25, wherein the oligonucleotide sequences are parallel.

30. The test kit according to claim 25, wherein the test kit further comprises at least one pair of oligonucleotide sequences attached to a solid support, one of said oligonucleotide sequences representing a full site and one of said oligonucleotide sequences representing an empty site thereby allowing co-dominant scoring.

31. The test kit according to claim 25, wherein the solid support comprises at least one membrane, filter, slide, plate, chip, dish or microwell composed of material selected from the group consisting of glass, plastics, nitrocellulose, nylon, polyacrylic acids and silicons.

32. The test kit according claim 25, wherein the test kit comprises elements selected from a group consisting of reagents, labels, washing buffers, end protecting reagents and instructions for use.

33. The test kit according claim 25, wherein the oligonucleotide sequences have a size allowing a formation of a stable hybridization product between the solid support attached oligonucleotide and the sample DNA.

34. The test kit according to claim 25, wherein the oligonucleotide sequences are end-protected.

35. The test kit according to claim 25, wherein the recording treatments are reversible allowing the solid support to be returned to its original state for reuse.

36. The method according to claim 1 for distinguishing any organism differing in at least one integration site of at least one mobile element (ME) integration site in any given genomic position.

37. The test kit according to claim 25 for distinguishing any organism differing in at least one integration site of at least one mobile element (ME) integration site in any given genomic position.

38. The method according to claim 1 for genotyping, phylogenetic studies, parenthood determinations, forensic science, human medical diagnostics, haplotyping, and pedigree analysis and in plant and animal breeding by demonstrating genetic identity, genetic diversity, genomic variation or polymorphism and particularly co-dominant scoring.

39. The test kit according to claim 25 for genotyping, phylogenetic studies, parenthood determinations, forensic science, human medical diagnostics, haplotyping, and pedigree analysis and in plant and animal breeding by demonstrating genetic identity, genetic diversity, genomic variation or polymorphism and particularly co-dominant scoring.

40. The method according to claim 1 for assured and accelerated breeding.

41. The test kit according to claim 25 for assured and accelerated breeding.
